SEQUENCE LISTING (12 sequences)

SEQ ID NO: 1 - DNA, 52 bp, Homo sapiens
  misc_feature (27)..(27): n = C or G
ctgcccttagtccctggcagactcctntgagctctttagtttatccttctaa

SEQ ID NO: 2 - DNA, 52 bp, Homo sapiens
  misc_feature (27)..(27): n = A or T
ttgaaagaaagtgccagctcctcaatnaaaacatgctcgaggcagacctacc

SEQ ID NO: 3 - DNA, 52 bp, Homo sapiens
  misc_feature (27)..(27): n = A or G
tttctcacatggtcatcagagtcacancgtatcctcagacttcagcagagca

SEQ ID NO: 4 - DNA, 52 bp, Homo sapiens
  misc_feature (27)..(27): n = G or T
gctagcgaaaccagagccacatgatantgatgctttacagtgagcttctgtc

SEQ ID NO: 5 - DNA, 52 bp, Homo sapiens
  misc_feature (27)..(27): n = A or G
aaagtggcttgtccagaatcacgctcngtgaatactaaagaggcatcacttt

SEQ ID NO: 6 - DNA, 52 bp, Homo sapiens
  misc_feature (27)..(27): n = A or G
gattaccagaggagggttgagaaaccncagagttttgaaaactttgggtcag

SEQ ID NO: 7 - DNA, 52 bp, Homo sapiens
  misc_feature (27)..(27): n = C or G
tatatacctgtcccactccactccccnacgcggcaaaccagcccttttatgg

SEQ ID NO: 8 - DNA, 51 bp, Homo sapiens
  misc_feature (27)..(27): n = A or G
tgcagagcctgaaggagaagtctgggntggggcccgggccaggattcggca

SEQ ID NO: 9 - DNA, 52 bp, Homo sapiens
  misc_feature (27)..(27): n = C or T
tagggtggtaaaatgctgtttgtctcnccgtccagggtagagccaggcaagg

SEQ ID NO: 10 - DNA, 52 bp, Homo sapiens
  misc_feature (27)..(27): n = A or G
ttccttatctgtggaatgggcctcatnacccccctcttgcaggactgtactg

SEQ ID NO: 11 - DNA, 1867 bp, Homo sapiens
cacatagctcagttcccataaaagggctggtttgccgcgtcggggagtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtgaaaaaaaaaaaaaaaaaaaaaaaaaaaaaa

SEQ ID NO: 12 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

(19) United States
(12) Patent Application Publication
(10) Pub. No.: US 2011/0177963 A1
(43) Pub. Date: Jul. 21, 2011

(54) Variation in the CHI3L1 Gene Influences Serum YKL-40 Levels, Asthma Risk and Lung Function

(75) Inventors: Geoffrey L. Chupp, Madison, CT (US); Jack A. Elias, Woodbridge, CT (US); Carole Ober, Chicago, IL (US)

(73) Assignees: Yale Unversity; The University of Chicago

(21) Appl. No.: 12/744,987

(22) PCT Filed: Nov. 26, 2008
(86) PCT No.: PCT/US08/85036

§ 371 Date: Apr. 7, 2011

Related U.S. Application Data
(60) Provisional application No. 61/004,548, filed on Nov. 28, 2007.

(51) Int. Cl. C40B 30/04; C12Q 1/68; G01N 33/559; G01N 33/53

(52) U.S. Cl. 506/9; 435/6.1; 435/6.12; 435/6.11; 436/515; 435/7.92

(57) ABSTRACT

The present invention is based on the discovery that a single nucleotide polymorphism (SNP) present the chitinase 3-like 1 gene (CHI3L1) encoding YKL-40 or a regulatory domain of the CHI3L1 gene, is associated with elevated YKL-40 levels, as well as an increased risk for developing a lung disorder, including asthma, bronchial hyperresponsivity, and/or reduced lung function.

### BACKGROUND OF THE INVENTION

Asthma is an inflammatory disease of the airways characterized by chronic respiratory symptoms and variable airflow obstruction that affects ˜7% of the U.S. population and millions of individual worldwide.

Chitinases are evolutionarily conserved proteins that mediate airway inflammation in mouse models of asthma (Zhu et al., 2004, Science 304:1678-1682). The chitinase-like protein YKL-40 lacks chitinase activity but binds ubiquitously expressed chitin and has been implicated in inflammation and tissue remodeling (Johansen et al., 2006, Dan. Med. Bull. 53:172-209; Johansen et al., 1993, Br. J. Rheumatol. 32:949-955; Johansen et al., 1992, J. Bone Min. Res. 7:501-512; Hakala et al., 1993, J. Biol. Chem. 268:25803-25810; Kelleher et al., 2005, J. Hepatol. 43:78-84). Serum YKL-40 levels are elevated in patients with asthma and circulating YKL-40 levels are correlated with asthma severity, thickness of the subepithelial basement membrane, and pulmonary function, suggesting that circulating YKL-40 levels are a biomarker for asthma (Chupp et al., 2007, N. Engl. J. Med. 357:2016-2027). The YKL-40 protein is encoded by the chitinase 3-like 1 gene CHI3L1, and single-nucleotide polymorphisms (SNPs) in the CHI3L1 promoter have been associated with elevated serum YKL-40 levels (Kruit et al., 2007, Respir. Med. 101:1563-1571; Zhao et al., 2007, Am. J. Hum. Genet. 80:12-18), differential gene expression (Zhao et al., 2007, Am. J. Hum. Genet. 80:12-18) and transcript levels (Dixon et al., 2007, Nature Genetics 39:1202-1207), and a higher risk of schizophrenia (Zhao et al., 2007, Am. J. Hum. Genet. 80:12-18).

There exists in the art a need to identify genes that affect serum YKL-40 levels as well as variations in genes that influence the risk of asthma, bronchial hyperresponsiveness, and are associated with reduced lung function. The present invention meets this need.

### SUMMARY OF THE INVENTION

The invention includes a method of identifying a human subject at-risk of developing a lung disorder, the method comprising obtaining a body sample from the subject; and, detecting at least one chromosomal variation in the CHI3L1 gene in the body sample, where if at least one chromosomal variation is detected in the gene, then the subject is at-risk of developing a lung disorder, where the lung disorder is selected from the group consisting of asthma, bronchial hyper-responsiveness, and reduced lung function. In one aspect, the body sample is selected from the group consisting of a tissue, a cell, and a bodily fluid. In another aspect, the detecting is performed using an assay selected from the group consisting of a PCR assay, a sequencing assay, an assay using a probe array, an assay using a gene chip, and an assay using a microarray. In still another aspect, the chromosomal variation is a −131 C→G in the promoter region of the CHI3L1 gene, defined by rs4950928 (SEQ ID NO:7).

Another embodiment of the invention includes a method of identifying a human subject at-risk of developing lung disorder, the method comprising: obtaining a body sample from the subject; detecting at least one disrupted transcript of the CHI3L1 gene in the body sample, where if at least one disrupted transcript is detected in the gene, then the subject is at-risk of developing a lung disorder, where the lung disorder is selected from the group consisting of asthma, bronchial hyperresponsiveness, and reduced lung function. In one aspect, the body sample is selected from the group consisting of a tissue, a cell, and a bodily fluid. In another aspect, the detecting is performed using an assay to assess the level of CHI3L1 mRNA, YKL-40 mRNA, or a combination thereof, in the body sample. In one aspect, the assay is selected from the group consisting of a Northern blot hybridization assay, an in situ hybridization assay, and a reverse transcriptase PCR assay. In another aspect, the detecting is performed using an assay to assess the level of CHI3L1 protein, YKL-40 protein, or a combination thereof, in the body sample. In still another aspect, the assay is selected from the group consisting of a Western blot assay, a radioimmunoassay (RIA), an immunoassay, a chemiluminescent assay, and an enzyme-linked immunosorbent assay (ELISA).

Yet another embodiment of the invention includes a method of identifying a human subject afflicted with asthma likely to benefit from treatment with Omalizumab, the method comprising obtaining a body sample from the subject; and, detecting YKL-40 expression in the body sample, where if YKL-40 expression in the sample is elevated relative to a control sample, then the subject is identified as likely to benefit from treatment with Omalizumab. In one aspect, the body sample is selected from the group consisting of a tissue, a cell, and a bodily fluid. In another aspect, the detecting is performed using an assay for YKL-40 mRNA. In another aspect, the assay is selected from the group consisting of a Northern blot hybridization assay, an in situ hybridization assay, and a reverse transcriptase PCR assay. In still another aspect, the detecting is performed using an assay for YKL-40 protein. In another aspect, the assay is selected from the group consisting of a Western blot assay, a radioimmunoassay (RIA), an immunoassay, a chemiluminescent assay, and a enzyme-linked immunosorbent assay (ELISA).

Still another embodiment of the invention includes a method of monitoring the efficacy of a therapeutic composition administered to a human subject for the treatment of asthma, the method comprising obtaining at least one body sample from the subject; and, detecting YKL-40 expression in the body sample, where if the YKL-40 expression in the sample remains elevated relative to a control sample after the composition is administered to the subject, then the composition is not efficacious for treating the subject. In one aspect, the body sample is selected from the group consisting of a tissue, a cell, and a bodily fluid. In another aspect, the detecting is performed using an assay for YKL-40 mRNA. In another aspect, the assay is selected from the group consisting of a Northern blot hybridization assay, an in situ hybridization assay, and a reverse transcriptase PCR assay. In yet another aspect, the detecting is performed in an assay for YKL-40 protein. In still another aspect, the assay is selected from the group consisting of a Western blot assay, a radioimmunoassay (RIA), an immunoassay, a chemiluminescent assay, and an enzyme-linked immunosorbent assay (ELISA).

Another embodiment of the invention includes a method of identifying a human subject afflicted with a refractory lung disorder, the method comprising obtaining a body sample from the subject; and, detecting at least one chromosomal variation in the CHI3L1 gene in the body sample, where if at least one chromosomal variation is detected in the gene, then the subject is identified as having a refractory lung disorder, where the refractory lung disorder is selected from the group consisting of refractory asthma, refractory bronchial hyperresponsiveness, and refractory reduced lung function. In one aspect, the body sample is selected from the group consisting of a tissue, a cell, and a bodily fluid. In another aspect, the detecting is performed in an assay selected from the group consisting of a PCR assay, a sequencing assay, an assay using a probe array, an assay using a gene chip, and an assay using a microarray. In another aspect, the chromosomal variation is a −131 C→G in the promoter region of said CHI3L1 gene, defined by rs4950928 (SEQ ID NO:7).

Still another embodiment of the invention includes a method of identifying a human subject afflicted with a refractory lung disorder, the method comprising obtaining a body sample from said subject; and detecting at least one disrupted transcript of the CHI3L1 gene in the body sample, where if at least one disrupted transcript is detected in the gene, then the subject is identified as having a refractory lung disorder, where the refractory lung disorder is selected from the group consisting of refractory asthma, refractory bronchial hyperresponsiveness, and refractory reduced lung function. In one aspect, the body sample is selected from the group consisting of a tissue, a cell, and a bodily fluid. In another aspect, the detecting is performed in an assay for CHI3L1 mRNA, YKL-40 mRNA, or a combination thereof in the body sample. In yet another aspect, the assay is selected from the group consisting of a Northern blot hybridization assay, an in situ hybridization assay, and a reverse transcriptase PCR assay. In still another aspect, the detecting is performed in an assay for CHI3L1 protein, YKL-40 protein or a combination thereof, in said body sample. In another aspect, the assay is selected from the group consisting of a Western blot assay, a radioimmunoassay (RIA), an immunoassay, a chemiluminescent assay, and an enzyme-linked immunosorbent assay (ELISA).

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph depicting depicting the level of HcGP-39/YKL-40 levels measured in serum of normal and asthmatic volunteers as part of the Yale patient cohort.

FIG. 2 is a graph depicting the relative increase in HcGP-39/YKL-40 level measured in serum as a function of disease severity in patients categorized as having mild, moderate or severe asthma as part of the Yale cohort.

FIG. 3 is a graph depicting the relative increase in HcGP-39/YKL-40 level measured in serum as a function of disease severity in patients categorized as having mild, moderate or severe asthma as part of the Wisconsin cohort.

FIG. 4 is a graph depicting the relative increase in HcGP-39/YKL-40 level measured in serum as a function of disease severity in patients categorized as having mild, moderate or severe asthma as part of the Paris cohort.

FIG. 5, comprising FIG. 5A through FIG. 5E, is a series of images depicting expression of YKL-40 protein in bronchial biopsies obtained from the Paris patient cohort. FIG. 10A depicts YKL-40 immunostaining in a biopsy obtained from a non-asthmatic patient. FIG. 10B-E depict YKL-40 immunostaining biopsies obtained from asthmatic patients. FIG. 10D and FIG. 10E depict YLK-40 immunostaining in a lung biopsy obtained from asthmatic patients characterized as having a severe form of asthma.

FIG. 6 is a graph depicting YKL-40 expression levels in cells obtained from the lung of normal and asthmatic patients.

FIG. 7 is a graph depicting the correlation of HcGP-30/YKL-40 expression in cells from lung with HcGP-30/YKL-40 expression in serum.

FIG. 8 is a schematic diagram depicting the linkage disequilibrium (r²) among SNPs in HapMap CEPH samples (of persons of European ancestry collected by the Centre d'Etude du Polymorphisme Humain) from 201,416,807 bp to 201,436,499 bp (Haploview). SNPs typed in the Hutterites and the SNP typed in the case and control populations (—131C→G) are indicated by black rectangles. SNPs in the linkage-disequilibrium plot are equally spaced across the region (and thus are not to physical scale).

FIG. 9, comprising FIG. 9A through FIG. 9D, is a series of graphs depicting serum YKL-40 level, asthma prevalence, and lung-function measures in Hutterites, according to −131C→G Genotype (rs4950928). All measures differed significantly among the three genotypes. FIG. 9A shows the mean natural-log-transformed serum YKL-40 levels (P=1.1×10⁻¹³ by the general two-allele model). FIG. 9B shows asthma prevalence among 554 Hutterites (P=0.047 by the case-control quasi-likelihood test). FIG. 9C shows the mean percent of the predicted forced expiratory volume in 1 second (FEV₁) (P=0.046 by the general two-allele model). FIG. 9D shows the mean ratio of FEV₁ to forced vital capacity (FVC) (P=0.002 by the general two-allele model).

FIG. 10 is a graph depicting mean serum YKL-40 levels in the Childhood Origins of Asthma Cohort, according to age and −131C→G Genotype (rs4950928). P values were calculated for the differences in mean natural-log-transformed serum YKL-40 levels among the three genotype groupings by means of an analysis of variance. Vertical bars indicate standard errors.

FIG. 11 is a graph depicting the relationship between rs4950928 allele and HcGP39/YKL-40 levels measured in serum.

FIG. 12 is a graph depicting CHI3L1 mRNA expression in non-asthmatic (control) and asthmatic (case) patients.

FIG. 13 is a graph depicting a change in YKL-40 levels in subjects afflicted with asthma that are treated with omalizumab.

FIG. 14 is a graph depicting YKL-40 levels in a subject before and after treatment with omalizumab.

### DETAILED DESCRIPTION OF THE INVENTION

The present invention is based on the discovery that a single nucleotide polymorphism (SNP) present the chitinase 3-like 1 gene (CHI3L1) encoding YKL-40 or a regulatory domain of the CHI3L1 gene, is associated with elevated YKL-40 levels, as well as an increased risk for developing a lung disorder, including asthma, bronchial hyperresponsivity, and/or reduced lung function. In particular, an allele of the SNP rs4950928, identified herein as −131C→G, is a marker for a human subject at risk for developing a more severe form of asthma, bronchial hyperresponsivity, and/or reduced lung function that is refractory to a standard treatment regimen.

### Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The phrase “body sample” as used herein, is intended any sample comprising a cell, a tissue, or a bodily fluid in which expression of a CHI3L1 gene or CHI3L1 gene product can be detected. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

The phrase “at-risk” as used herein refers to a subject with a greater than average likelihood of developing asthma, bronchial hyperresponsivity, or reduced lung function.

As used herein, an “allele” is one of several alternate forms of a gene or non-coding regions of DNA that occupy the same position on a chromosome.

A “biomarker” or “marker” of the invention is any detectable molecule, nucleic acid, protein, peptide, compound, or agent present in a body sample obtained from a subject that identifies the subject as being at-risk for asthma, bronchial hyperresponsivity, or reduced lung function. A biomarker of the invention may further comprise any detectable chromosomal variation, including but limited to a single nucleotide polymorphism (SNP), that contributes to a subject being at-risk for asthma, bronchial hyperresponsivity, or reduced lung function. A chromosomal variation may be detected at either the nucleic acid or protein level.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Substantially complementary to” refers to probe or primer sequences which hybridize to the sequences listed under stringent conditions and/or sequences having sufficient homology with test polynucleotide sequences, such that the allele specific oligonucleotide probe or primers hybridize to the test polynucleotide sequences to which they are complimentary.

The term “disease,” as used herein, refers to any deviation from or interruption of the normal structure or function of any body part, organ, or system that is manifested by a characteristic set of symptoms and signs and whose etiology, pathology, and prognosis may be known or unknown.

A “refractory disease,” as used herein refers to a disease that has not responded to or has ceased responding to an initial therapy or to convention compositions and therapeutic regimens used to treat that disease.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Sequence variation” as used herein refers to any difference in nucleotide sequence between two different oligonucleotide or polynucleotide sequences.

“Polymorphism” as used herein refers to a sequence variation in a gene which is not necessarily associated with pathology.

“Single nucleotide polymorphism” as used herein, is a DNA sequence variation occurring when a single nucleotide (A,T,C, or G) in the genome differs between members of a species, or between paired chromosomes in an individual, and both versions are observed in the general population at a frequency greater than 1%. Almost all common SNPs have only two alleles. Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are nonsynonymous. A nonsynonymous change may either be missense or “nonsense”, where a missense change results in a different amino acid, while a nonsense change results in a premature stop codon. SNPs that are not in protein-coding regions may still have consequences for gene splicing, transcription factor binding, or the sequence of non-coding RNA. Variations in the DNA sequences of humans, e.g. SNPs, can affect how humans develop diseases and respond to pathogens, chemicals, drugs, vaccines, and other agents.

“Mutation” as used herein refers to an altered genetic sequence which results in the gene coding for a non-functioning protein or a protein with substantially reduced or altered function. Generally, a deleterious mutation is associated with pathology or the potential for pathology.

“Allele specific detection assay” as used herein refers to an assay to detect the presence or absence of a predetermined sequence variation in a test polynucleotide or oligonucleotide by annealing the test polynucleotide or oligonucleotide with a polynucleotide or oligonucleotide of predetermined sequence such that differential DNA sequence based techniques or DNA amplification methods discriminate between normal and mutant.

“Sequence variation locating assay” as used herein refers to an assay that detects a sequence variation in a test polynucleotide or oligonucleotide and localizes the position of the sequence variation to a subregion of the test polynucleotide, without necessarily determining the precise base change or position of the sequence variation.

The “regulatory region” of a gene, or “regulatory sequence”, as used herein, can be divided into cis-regulatory (or cis-acting) elements and trans-regulatory (or trans-acting) elements. The cis-regulatory elements are the binding sites of transcription factors which are the proteins that, upon binding with cis-regulatory elements, can affect (either enhance or repress) transcription. The trans-regulatory elements are the DNA sequences that encode transcription factors. The cis-acting elements may be divided into four types: promoters, enhancers, silencers, and response elements. A promoter is the DNA element where the transcription initiation takes place. An enhancer is the element that, upon binding with transcription factors, can enhance transcription. The transcription factors that bind to enhancers are called transcriptional activators. A silencer is the element that, upon binding with transcription factors, can repress transcription. The transcription factors that bind to silencers are called repressors. A response element is the recognition site of certain transcription factors.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

By the term “specifically binds,” as used herein, is meant an antibody which recognizes and binds a biomarker or fragment thereof, but does not substantially recognize or bind other molecules in a sample.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, an “instructional material” includes a publication, a recording, a diagram, or any other medium of expression, which can be used to communicate the usefulness of the nucleic acid, peptide, and/or composition of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container, which contains the nucleic acid, peptide, chemical compound and/or composition of the invention or be shipped together with a container, which contains the nucleic acid, peptide, chemical composition, and/or composition. Alternatively, the instructional material may be shipped separately from the container with the intention that the instructional material and the compound be used cooperatively by the recipient.

### Description:

The present invention is based in part on the discovery of a single nucleotide polymorphism within the CHI3L1 gene, or a regulatory domain thereof, that functions as a biomarker for asthma, bronchial hyperresponsivity, or decreased lung function in a human subject.

In one embodiment, a biomarker of the invention comprises a detectable chromosomal variation, including, but limited to a single nucleotide polymorphism (SNP), that contributes to a subject being at-risk for asthma, bronchial hyperresponsivity, or reduced lung function. A chromosomal variation may be detected at either the nucleic acid or protein level.

In another embodiment, a biomarker of the invention comprises a disrupted gene product that contributes to a subject being at-risk for asthma, bronchial hyperresponsivity, or decreased lung function. A disrupted gene product of the invention comprises any gene product that is a variant of a normal gene product or is expressed at abnormal levels such that the disrupted gene product cannot fulfill the normal gene product's function, and thus, contributes to the etiology of asthma, bronchial hyperresponsivity, or reduced lung function. A disrupted gene product of the invention therefore includes a variant mRNA and/or a protein that contributes to the etiology of asthma, bronchial hyperresponsivity, or reduced lung function, especially asthma, bronchial hyperresponsivity, and/or reduced lung function that is refractory to conventional therapeutic compositions or treatment regimens. A disrupted gene product of the invention further includes a normal protein or mRNA that is expressed at aberrant levels, either at excess levels as compared to normal expression or at insufficient levels as compared to normal expression.

In one embodiment, a method of identifying a human subject at-risk of developing asthma, bronchial hyperresponsivity, or reduced lung function is provided. The method comprises obtaining a body sample from a subject at-risk of developing asthma, bronchial hyperresponsivity, or decreased lung function, and detecting at least one SNP in the CHI3L1 gene or regulatory sequence thereof in a body sample obtained from the subject that contributes to the etiology of asthma, bronchial hyperresponsivity, or reduced lung function. If at least one such SNP is detected, then the subject is at-risk of developing asthma, bronchial hyperresponsivity, or reduced lung function.

In one embodiment, invention includes a method of identifying a human subject at-risk of developing asthma, bronchial hyperresponsivity, or reduced lung function is provided. The method comprises obtaining a body sample from a subject at-risk of developing asthma, bronchial hyperresponsivity, or decreased lung function, and detecting the expression level of YKL-40, or a fragment thereof, in the body sample. If elevated levels of YKL-40 expression are detected in the sample relative to a control sample, then the subject is at-risk of developing asthma, bronchial hyperresponsivity, or reduced lung function.

In another embodiment, there is provided a method of identifying a human subject at-risk of developing refractory asthma, refractory bronchial hyperresponsivity, or refractory reduced lung function. The method comprises obtaining a body sample from the subject, and detecting at least one SNP in the CHI3L1 gene or regulatory sequence thereof in the body sample. If at least one such SNP is detected, then said subject is at-risk of developing refractory asthma, refractory bronchial hyperresponsivity, or refractory reduced lung function.

In yet another embodiment, there is included a method of identifying a human subject at-risk of developing refractory asthma, refractory bronchial hyperresponsivity, or refractory reduced lung function, where the method comprises obtaining a body sample from the subject and detecting the expression level of YKL-40, or a fragment thereof, in the body. If elevated levels of YKL-40 expression are detected in the sample relative to a control sample, then said subject is at-risk of developing refractory asthma, refractory bronchial hyperresponsivity, or refractory reduced lung function.

In another embodiment, the invention includes a method of identifying a human subject afflicted with asthma likely to benefit from a particular therapeutic composition or therapeutic regimen. In one aspect, a therapeutic composition useful in treating asthma, bronchial hyperresponsivity, and/or reduced lung function, especially refractory forms of these diseases, comprises Omalizumab (Genetech/Novartis, San Francisco, Calif.). The method comprises obtaining a body sample from a subject and detecting at least one SNP in the CHI3L1 gene or regulatory sequence thereof in the body sample. If at least one such SNP is detected, then the subject is likely to benefit from treatment with a therapeutic composition comprising Omalizumab.

In another embodiment, the invention includes a method of identifying a human subject afflicted with asthma likely to benefit from a particular therapeutic composition or therapeutic regimen. In one embodiment, a therapeutic composition useful in treating asthma, bronchial hyperresponsivity, and/or reduced lung function, especially refractory forms of these diseases, comprises Omalizumab. The method comprises obtaining a body sample from a subject and detecting YKL-40 expression in the body sample. If YKL-40 expression is elevated relative to a control sample, then the subject is likely to benefit from treatment with a therapeutic composition comprising Omalizumab.

In still another embodiment, there is provided a method of monitoring the efficacy of a therapeutic composition or therapeutic regimen administered to a human subject for the treatment of a lung disorder, including asthma, bronchial hyperresponsivity, and/or reduced lung function. The method comprises obtaining a body sample from a subject and detecting YKL-40 expression in the body sample. If YKL-40 expression is elevated relative to a control sample, or remains unchanged relative to a control sample, then the subject has not benefited from the therapeutic composition or therapeutic regimen.

The present invention identifies specific SNPs of the CHI3L1 gene as associated with elevated YKL-40 levels in a body sample (Table 1). In one embodiment, thepresenter is provided a method of identifying a human subject at-risk of developing asthma, bronchial hyperresponsivity, or reduced lung function. The method comprises detecting at least one SNP selected from the group consisting of rs2153101, rs946263, rs4950929, and rs4950928, wherein if the allele detected for a given SNP is associated with increased YKL-40 expression, then the subject is at-risk of developing asthma, bronchial hyperresponsivity, or reduced lung function.

In a preferred embodiment, there is provided a method that comprises detecting the SNP rs4950928 (SEQ ID NO. 7) in a body sample obtained from a human subject, wherein the allele detected for rs4950928 is the −131 C→G allele associated with asthma, bronchial hyperresponsivity, or reduced lung function. In one aspect, if the minor G allele of rs4950928 is detected, then the subject is identified as having a less severe form of disease. In another aspect, if the C allele of rs4950928 is detected, then the subject is identified as being at risk for, or having refractory asthma, refractory bronchial hyperresponsivity, and refractory decreased lung function. In still another aspect, if the C allele of rs4950928 is detected, then the subject is identified as a candidate for treatment with Omalizumab.

TABLE 1
Sequence variations of SNPs.

| | SEQ | |
|---|---|---|
| | ID | |
| SNP | NO. | Sequence |
| rs871799 | 1 | CTGCCCTTAGTCCCTGGCAGACTCCT[C/G] |
| | | TGAGCTCTTTAGTTTATCCTTCTAA |
| rs2153101* | 2 | TTGAAAGAAAGTGCCAGCTCCTCAAT[A/T] |
| | | AAAACATGCTCGAGGCAGACCTACC |
| rs946263* | 3 | TTTCTCACATGGTCATCAGAGTCACA[A/G] |
| | | CGTATCCTCAGACTTCAGCAGAGCA |
| rs4950929* | 4 | GCTAGCGAAACCAGAGCCACATGATA[G/T] |
| | | TGATGCTTTACAGTGAGCTTCTGTC |
| rs6691378 | 5 | AAAGTGGCTTGTCCAGAATCACGCTC[A/G] |
| | | GTGAATACTAAAGAGGCATCACTTT |
| rs10399805 | 6 | GATTACCAGAGGAGGGTTGAGAAACC[A/G] |
| | | CAGAGTTTTGAAAACTTTGGGTCAG |
| rs4950928*† | 7 | TATATACCTGTCCCACTCCACTCCCC[C/G] |
| | | ACGCGGCAAACCAGCCCTTTTATGG |
| rs1538372 | 8 | TGCAGAGCCTGAAGGAGAAGTCTGGG[A/G] |
| | | TGGGGCCCGGGCCAGGATTCGGCA |
| rs880633 | 9 | TAGGGTGGTAAAATGCTGTTTGTCTC[C/T] |
| | | CCGTCCAGGGTAGAGCCAGGCAAGG |
| rs2275352 | 10 | TTCCTTATCTGTGGAATGGGCCTCAT[A/G] |
| | | ACCCCCCTCTTGCAGGACTGTACTG |

Bracketed region of the sequence of an SNP depicts the alternative sequences for the two alleles that comprise a given SNP.
*SNPs that showed strongest associations with serum YKL-40 levels
†SNP that was also associated with asthma, bronchial hyperresponsivity and decreased lung function

A “control” or “control sample,” as used herein refers to a body sample obtained from a subject not at-risk of developing asthma, bronchial hyperresponsivity, or decreased lung function. In one embodiment, a control sample may be obtained from a single individual not afflicted with asthma, bronchial hyperresponsivity, or decreased lung function, or not at-risk of asthma, bronchial hyperresponsivity, or decreased lung function.

In another embodiment, a control sample may be obtained from an individual afflicted with asthma, bronchial hyperresponsivity, and/or decreased lung function where that individual is undergoing treatment. The control sample may be obtained from the same individual before that individual has begun a therapeutic treatment or regimen. In another embodiment, a sample may be obtained from an individual undergoing treatment for asthma, bronchial hyperresponsivity, and/or decreased lung function at different time points during the treatment. Samples obtained earlier in the treatment regimen may act as control samples for samples obtained later during the treatment regimen.

In another embodiment, the control sample may comprise a pooled sample containing body samples obtained from a population of subjects where those subjects have been identified as negative for asthma, bronchial hyperresponsivity, or decreased lung function, or not being at-risk for asthma, bronchial hyperresponsivity, or decreased lung function. It is understood that when the control sample is obtained from multiple samples, the marker expression level can be expressed as an arithmetic mean, median, mode, or other suitable statistical measure of marker expression level measured in each sample. Multiple control samples may be pooled, and the marker expression level of the pooled samples may be compared to the subject's body sample.

The invention may be practiced on any subject diagnosed with, or at-risk of developing asthma, bronchial hyperresponsivity, or decreased lung function. Preferably the subject is a mammal, and more preferably a human.

### I. Detecting Single Nucleotide Polymorphisms (SNPs)

Methods for detecting a SNP associated with elevated YKL-40 expression, asthma, bronchial hyperresponsivity, and/or decreased lung function comprise any method or assay that interrogates the CHI3L1 gene. A number of assay formats known in the art are useful for detecting SNPs. These methods commonly involve nucleic acid binding, e.g., to filters, beads, chips and the like; and include hybridization assays, PCR, sequencing assays, or combinations thereof.

A. FP-TDI Method of Allele-specific Primer Extension

FP-TDI stands for template directed dye terminator incorporation assay with detection by fluorescence polarization. It is a single base primer extension assay couple with homogeneous FP detection (Chen et al., 1999, Genome Res. 9:492-498)

There are four key steps to a FP-TDI assay: template amplification by PCR protocol; PCR product clean-up; single-base primer extension using a primer that anneals one base shy of the polymorphic site, and

Template-directed primer extension is a dideoxy chain-fluorophore labeled terminators; FP reading and data analysis terminating DNA-sequencing protocol designed to ascertain the nature of the one base immediately 3′ to the sequencing primer that is annealed to the target DNA immediately upstream from the polymorphic site. In the presence of DNA polymerase and the appropriate dideoxyribonucleoside triphosphate (ddNTP), the primer is extended specifically by one base as dictated by the target DNA sequence at the polymorphic site. By determining which ddNTP is incorporated, the alleles present in the target DNA can be inferred.

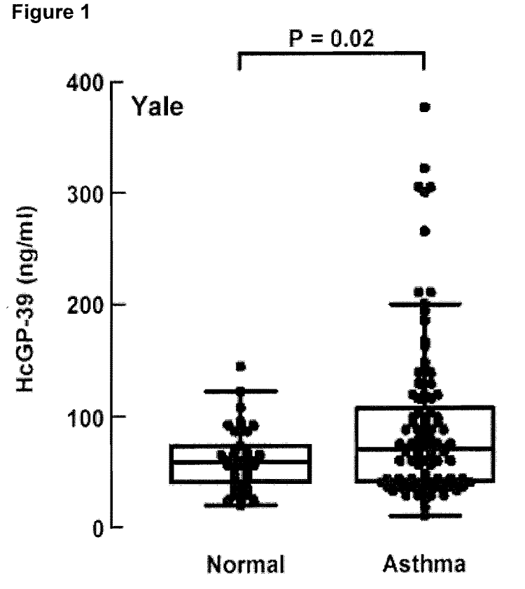

Fluorescence polarization (FP) is a popular technique designed for homogeneous, high throughput assays based on the observation that when a fluorescent molecule is excited by plane-polarized light, it emits polarized fluorescent light into a fixed plane if the molecules remain stationary between excitation and emission. Because the molecule rotates and tumbles in space, however, FP is not observed fully by an external detector. The FP of a molecule is proportional to the molecule's rotational relaxation time (the time it takes to rotate through an angle of 68.5°), which is related to the viscosity of the solvent, absolute temperature, molecular volume, and the gas constant. Therefore, if the viscosity and temperature are held constant, FP is directly proportional to the molecular volume, which is directly proportional to the molecular weight. If the fluorescent molecule is large (with high molecular weight), it rotates and tumbles more slowly in space and FP is preserved. If the molecule is small (with low molecular weight), it rotates and tumbles faster and FP is largely lost (depolarized) (FIG. 1). The FP phenomenon has been used to study protein-DNA and protein-protein interactions (Checovich et al., 1995, Nature 375:254-256; Heyduk et al., 1996, Meth. Enzymol. 274:492-503), DNA detection by strand displacement amplification (Walker et al. 1996), and in genotyping by hybridization (Gibson et al., 1997, Clin. Chem. 43:1336-1341). Currently, >50 fluorescence polarization immunoassays (FPIA) are commercially available, many of which are routinely used in clinical laboratories for the measurement of therapeutics, metabolites, and drugs of abuse in biological fluids (Checovich et al., 1995, Nature 375:254-256).

FP is expressed as the ratio of fluorescence detected in the vertical and horizontal axes and, therefore, is independent of the fluorescence intensity. This is a clear advantage over other fluorescence detection methods in that as long as the fluorescence is above detection limits of the instrument used, FP is a reliable measure. The degree of FP increases more or less linearly up to 10,000 Daltons in molecular mass before it levels off. Because a nucleotide bearing a fluorescent molecule has a molecular mass of ˜1000 Daltons and a fluorescent 25- to 30-mer is ˜10,000 Daltons, FP is well suited as a detection method for the primer extension reaction.

In template-directed dye-terminator incorporation assay with FP detection (FP-TDI assay), the sequencing primer is an unmodified primer with its 3′ end immediately upstream from the polymorphic or mutation site. When incubated in the presence of ddNTPs labeled with different fluorophores, the allele-specific dye-labeled ddNTP is incorporated onto the TDI primer in the presence of DNA polymerase and target DNA. The genotype of the target DNA molecule can be determined simply by exciting the fluorescent dye in the reaction and determining whether a change in FP is observed.

B. Allele Specific Hybridization

Also known as allele specific oligonucleotide hybridization (ASO), this protocol relies on distinguishing between two DNA molecules differing by one base by hybridization. Fluorescence labeled PCR fragments are applied to immobilized oligonucleotides representing SNP sequences. After stringent hybridization and washing conditions, fluorescence intensity is measured for each SNP oligonucleotide.

C. Primer Extension

In the single base extension approach, the target region is amplified by PCR followed by a single base sequencing reaction using a primer that anneals one base shy of the polymorphic site. Several detection methods have been described. One can label the primer and apply the extension products to gel electrophoresis. Or the single base extension product can be broken down into smaller pieces and measured by Mass Spectrometry. The most popular detection method involves fluorescence labeled, dideoxynucleotide terminators that stop the chain extension.

D. Allele Specific Oligonucleotide Ligation

By designing oligonucleotides complementary to the target sequence, with the allele-specific base at its 3′-end or 5-′end, one can determine the genotype of the PCR amplified target sequence by determining whether an oligonucleotide complementary to the DNA sequencing adjoining the polymorphic site is ligated to the allele-specific oligonucleotide or not.

E. Sequencing

Sequencing is the procedure of choice for SNP discovery. The most common forms of sequencing are based on primer extension using either a) dye-primers and unlabeled terminators or b) unlabeled primers and dye-terminators. The products of the reaction are then separated using electrophoresis using either capillary electrophoresis or slab gels.

### II. Detection of a Disrupted Gene Product

In another embodiment, the present invention identifies a disrupted product of the CHI3L1 gene as a biomarker for a subject at-risk of developing asthma, bronchial hyperresponsivity, and/or reduced lung function. The gene product may be an mRNA or a protein variant. A disrupted gene product may also be a protein, peptide, or fragment thereof that is expressed at aberrant levels. One such gene product is YKL-40 mRNA or protein (SEQ ID NO. 12). The nucleic acid sequence encoding YKL-40 protein is recited in SEQ ID NO. 11. Accordingly, elevated YKL-40 levels are a biomarker for asthma, bronchial hyperresponsivity, or reduced lung function.

### A. Protein Assays

In another embodiment of the invention, disruption of a gene product is detected at the protein level using antibodies specific for biomarker proteins of the invention, including YKL-40 (SEQ ID NO. 12) or a fragment thereof.

The method comprises obtaining a body sample from a patient, contacting the body sample with at least one antibody directed to a biomarker. One of skill in the art will recognize that the immunocytochemistry method described herein below is performed manually or in an automated fashion.

When the antibody used in the methods of the invention is a polyclonal antibody (IgG), the antibody is generated by inoculating a suitable animal with a biomarker protein, peptide or a fragment thereof. Antibodies produced in the inoculated animal which specifically bind the biomarker protein are then isolated from fluid obtained from the animal. Biomarker antibodies may be generated in this manner in several non-human mammals such as, but not limited to goat, sheep, horse, rabbit, and donkey. Methods for generating polyclonal antibodies are well known in the art and are described, for example in Harlow, et al. (1998, In: Using Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988, Blood, 72:109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of biomarker may be prepared using the techniques described in Harlow, et al. (1988, In: Antibodies, A Laboratory Manual, Cold Spring Harbor, N.Y.).

Samples may need to be modified in order to render the biomarker antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO 51699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a biomarker of interest is then incubated with the sample.

Techniques for detecting antibody binding are well known in the art. Antibody binding to a biomarker of interest may be detected through the use of chemical reagents that generate a detectable signal that corresponds to the level of antibody binding and, accordingly, to the level of biomarker protein expression. In one of the preferred immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), may be used to practice the present invention.

In one particular immunocytochemistry method of the invention, antibody binding to a biomarker is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., biomarker overexpression). Alternatively, samples may be reviewed via automated microscopy or by personnel with the assistance of computer software that facilitates the identification of positive staining cells.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the invention, there also exist in the art video-microscopy and software methods for the quantitative determination of an amount of multiple molecular species (e.g., biomarker proteins) in a biological sample, wherein each molecular species present is indicated by a representative dye marker having a specific color. Such methods are also known in the art as colorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 and U.S. patent application Ser. No. 10/057,729 to Marcelpoil., incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for the biomarker proteins of interest. Methods for making antibodies and for selecting appropriate antibodies are known in the art. See, for example, Celis, J. E. ed. (2006, Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined in reference to the type of biological sample being tested. In particular embodiments, appropriate antibody titers for use cytology preparations are determined by initially testing various antibody dilutions on formalin-fixed paraffin-embedded normal tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody is then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the invention will vary depending on such factors as time for binding, level of specificity of the antibody for the biomarker protein, and method of body sample preparation. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

### Immunoassays

Immunoassays, in their simplest and most direct sense, are binding assays.

Certain preferred immunoassays are the various types of enzyme linked immunosorbent assays (ELISA) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the biomarker proteins of the invention are immobilized onto a selected surface exhibiting protein affinity, such as a well in a polystyrene microtiter plate. Then, a test composition suspected of containing the biomarker antigen, such as a clinical sample, is added to the wells. After binding and washing to remove non-specifically bound immunecomplexes, the bound antibody may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the biomarker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the biomarker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immunecomplex can be detected with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

B. mRNA Assays

In another embodiment of the invention, disruption of a gene product is detected at the mRNA level. Nucleic acid-based techniques for assessing mRNA expression are well known in the art and include, for example, determining the level of biomarker mRNA in a body sample. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

Isolated mRNA as a biomarker can be detected in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

An alternative method for detecting biomarker mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan.®. System). Such methods typically use pairs of oligonucleotide primers that are specific for the biomarker of interest. Methods for designing oligonucleotide primers specific for a known sequence are well known in the art.

Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

### Microarray

In one embodiment of the invention, microarrays are used to detect biomarker expression in a biological sample. Microarrays are particularly well suited for this purpose because of their reproducibility. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

Nucleic acids which code for a biomarker can be placed in an array on a substrate, such as on a chip (e.g., DNA chip or microchips). These arrays also can be placed on other substrates, such as microtiter plates, beads or microspheres. Methods of linking nucleic acids to suitable substrates and the substrates themselves are described, for example, in U.S. Pat. Nos. 5,981,956; 5,922,591; 5,994,068 (Gene Logic's Flow-thru ChipO Probe ArraysO); U.S. Pat. Nos. 5,858,659, 5,753,439; 5,837,860 and the FlowMetrix technology (e.g., microspheres) of Luminex (U.S. Pat. Nos. 5,981,180 and 5,736,330).

The methods of the present invention do not require that the target nucleic acid contain only one of its natural two strands. Thus, the methods of the present invention may be practiced on either double-stranded DNA (dsDNA), or on single-stranded DNA (ssDNA) obtained by, for example, alkali treatment of native DNA. The presence of the unused (non-template) strand does not affect the reaction.

Where desired, however, any of a variety of methods can be used to eliminate one of the two natural stands of the target DNA molecule from the reaction. Single-stranded DNA molecules may be produced using the ssDNA bacteriophage, M13 (Messing, 1983, Meth. Enzymol., 101: 20-78; see also, Sambrook, 2001, Molecular Cloning: A Laboratory Manuel, 3^{rd} ed. (Cold Spring Harbor Laboratory Press, Cold Spring Harbor, N.Y.).

Several alternative methods can be used to generate single-stranded DNA molecules. For example, Gyllensten, 1988, Proc. Natl. Acad. Sci. U.S.A., 85: 7652-6 and Mihovilovic, 1989, BioTechiques, 7: 14-6 describe a method, termed “asymmetric PCR,” in which the standard “PCR” method is conducted using primers that are present in different molar concentrations.

Other methods have also exploited the nuclease resistant properties of phosphorothioate derivatives in order to generate single-stranded DNA molecules (U.S. Pat. No. 4,521,509; Sayers, 1988, Nucl. Acids Res., 16: 791-802; Eckstein, 1976, Biochemistry 15: 1685-91; Ott, 1987, Biochemistry 26: 8237-41; see also, Sambrook, 2001, Molecular Cloning: A Laboratory Manuel, 3^{rd} ed. (Cold Spring Harbor Laboratory Press, Cold Spring Harbor, N.Y.).

The target nucleic acid is hybridized with the array and scanned. A target nucleic acid sequence, which includes one or more previously identified biomarkers, is amplified by well known amplification techniques, e.g., polymerase chain reaction (PCR). Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single biomarker, in preferred aspects of the invention, the arrays of the invention include multiple detection blocks, and are thus capable of analyzing multiple, specific biomarkers. For example, preferred arrays generally include from about 50 to about 4,000 different detection blocks with particularly preferred arrays including from 10 to 3,000 different detection blocks.

In alternate arrangements, it is generally understood that detection blocks may be grouped within a single array or in multiple, separate arrays so that varying, optimal conditions may be used during the hybridization of the target to the array. For example, it may often be desirable to provide for the detection of those polymorphisms that fall within G C rich stretches of a genomic sequence, separately from those falling in A T rich segments. This allows for the separate optimization of hybridization conditions for each situation.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

### Preparation of Nucleic Acid Probes

Using the sequence information provided herein, the nucleic acids may be synthesized according to a number of standard methods known in the art. Oligonucleotide synthesis, is carried out on commercially available solid phase oligonucleotide synthesis machines or manually synthesized using the solid phase phosphoramidite triester method described by Beaucage, 1981, Tetrahedron Letters, 22: 1859-1862.

Once a nucleic acid encoding a biomarker is synthesized, it may be amplified and/or cloned according to standard methods in order to produce recombinant polypeptides. Molecular cloning techniques to achieve these ends are known in the art. A wide variety of cloning and in vitro amplification methods suitable for the construction of recombinant nucleic acids are known to those skilled in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook, 2001, Molecular Cloning: A Laboratory Manuel, 3^{rd} ed. (Cold Spring Harbor Laboratory Press, Cold Spring Harbor, N.Y.

Once the nucleic acid for a biomarker is cloned, a skilled artisan may express the recombinant gene(s) in a variety of engineered cells. Examples of such cells include bacteria, yeast, filamentous fungi, insect (especially employing baculoviral vectors), and mammalian cells. It is expected that those of skill in the art are knowledgeable in the numerous expression systems available for expressing the biomarker proteins of the invention.

### Kits

Kits for practicing the methods of the invention are further provided. By “kit” is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a unit for performing the methods of the present invention. Additionally, the kits may contain a package insert describing the kit and including instructional material for its use.

Positive and/or negative controls may be included in the kits to validate the activity and correct usage of reagents employed in accordance with the invention. Controls may include samples, such as tissue sections, cells fixed on glass slides, etc., known to be either positive or negative for the presence of the biomarker of interest. The design and use of controls is standard and well within the routine capabilities of those of ordinary skill in the art.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described along with the results of the experiments presented in these Examples.

### Experiment 1

### YKL-40 Expression is Elevated in the Circulation and Lung of Severe Asthmatics

### Subjects and Patient Cohorts:

A cross-sectional analysis was performed on samples from an established cohort of asthmatic subjects from the Yale Center for Asthma and Airways Disease (YCAAD). A second set of serum samples from the University of Wisconsin and a third set of samples from the University of Paris were also examined. In the Yale cohort the normal and asthmatic subjects were similar in demographic characteristics including age, sex and race. There were significant differences in factors known to be associated with asthma including a higher BMI (P=0.01), a history of atopy (P=0.001) and elevated IgE levels (P=0.001). When asthmatics were compared by disease severity, there were more African-American and Latino-American patients with severe, versus mild and moderate disease. Severe asthmatics had a history of more hospitalizations, intubations, rescue medication use, oral corticosteroid tapers, longer duration of asthma, and more severely compromised pulmonary function than the milder asthmatics. The characteristics of the University of Wisconsin and Paris populations had similar characteristics compared to the Yale cohort. In the former, comparisons on the basis of severity illustrated differences in age of asthma onset, asthma duration, hospitalizations, urgent care visits, inhaled corticosteroid dose, and pulmonary function that were comparable to the Yale cohort. In the Paris population, significant differences in the rates of atopy, levels of IgE, corticosteroid dose and lung function were noted with increasing asthma severity.

### Results:

The expression of YKL-40 in the airway and its relationship to serum YKL-40 levels was investigated in the Paris cohort. The recruitment of controls and asthmatics coincided, and similar methods were used at each center to recruit subjects from existing patient populations and the surrounding communities. Each center had its own criteria for controls, asthmatics, and asthma severity based on established guidelines. All subjects gave informed consent. Serum samples were obtained, aliquoted and used immediately or frozen at −80° C. Each patient with asthma was classified as mild, moderate or severe using severity criterion adopted from the American Thoracic Society (ATS) Workshop on Refractory Asthma and the classification from the National Asthma Education and Prevention Program (NAEPP). YKL-40 levels were measured using a commercially available enzyme-linked immunosorbent assay (ELISA) (YKL-40, Quidel, San Diego, Calif.; IgE, Pharmacia, Minneapolis, Minn. and Dade-Behring, Paris, France) and median values are presented. The minimum detection limit of the YKL-40 assay is 20 ng/ml. To confirm the specificity of the YKL-40 ELISA, the capture and detection antibodies were demonstrated to lack cross reactivity to other human chitinases, including AMCase, YKL-39 and chitotriosidase.

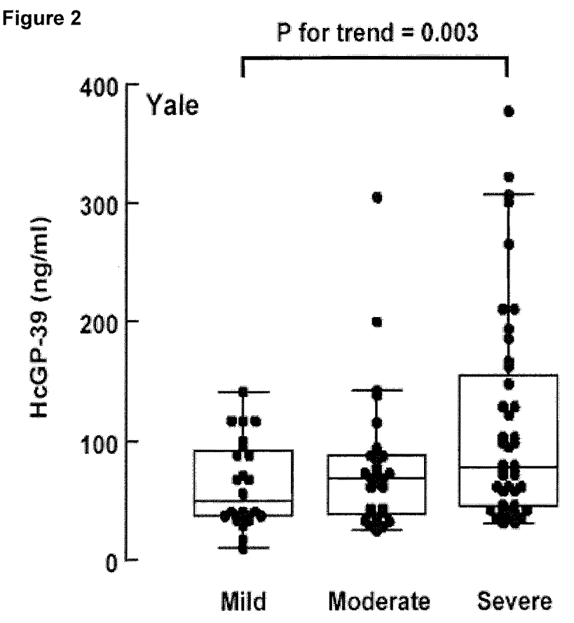

Serum YKL-40 Levels Correlate with Asthma and Asthma Severity: The levels of YKL-40 were measured in the sera from the Yale cohort. YKL-40 was readily appreciated in the serum from normal volunteers and was significantly higher in the serum from asthmatics [median (interquartile range) 58.3 ng/ml (40.0-73.3) versus 69.7 (40.0-107.1), P=0.02, FIG. 1]. Importantly, YKL-40 levels increased with disease severity, with the highest levels observed in refractory asthmatics, compared to moderate and mild asthmatics, respectively (P for trend=0.003, FIG. 2). The median (interquartile range) YKL-40 level in mild asthmatics was 49.11 ng/ml (36.7-94.2), 68.43 ng/ml (38.0-88.0) in moderate asthmatics, and 77.0 (44.6-158.4) in severe asthmatics. Interestingly, 42 asthmatics had 107 repeat measurements during the 4-year study interval (31 subjects had 2 measurements, 7 subjects had 3 measurements and 4 subjects had 4 measurements). The mean coefficient of variation was 37%. This was significantly less that other biomarkers we have evaluated (TARC and IP-10).

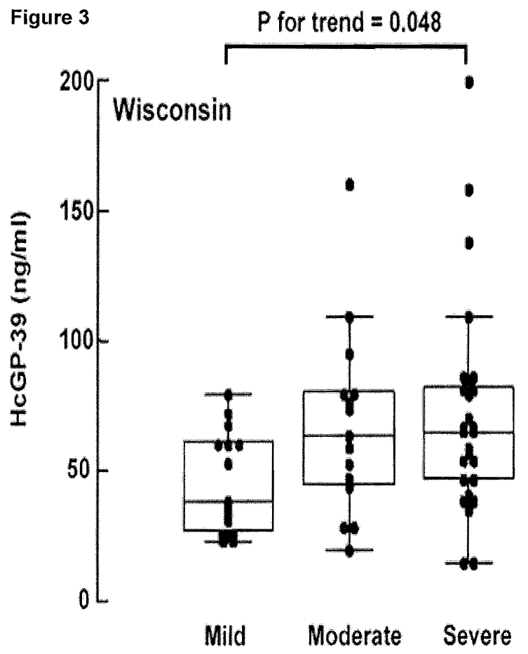

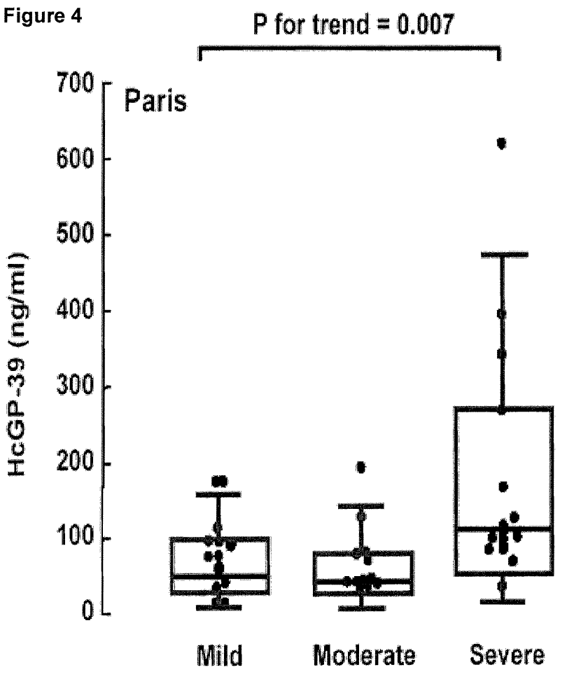

The levels of circulating YKL-40 and asthma severity were also evaluated in the University of Wisconsin and Paris cohorts (FIGS. 3 and 4). The association between asthma severity and the levels of circulating YKL-40 was evident in the Paris population [45.5 ng/ml (24.5-78.5), 41.0 ng/ml (25.0-67.0) and 94.0 ng/ml (72.0-181.5), P for trend=0.007] and the Wisconsin cohort [49.11 ng/ml (36.7-94.2), 68.43 ng/ml (38.0-88.0), and 77.0 ng/ml (44.6-158.4), P for trend<0.05].

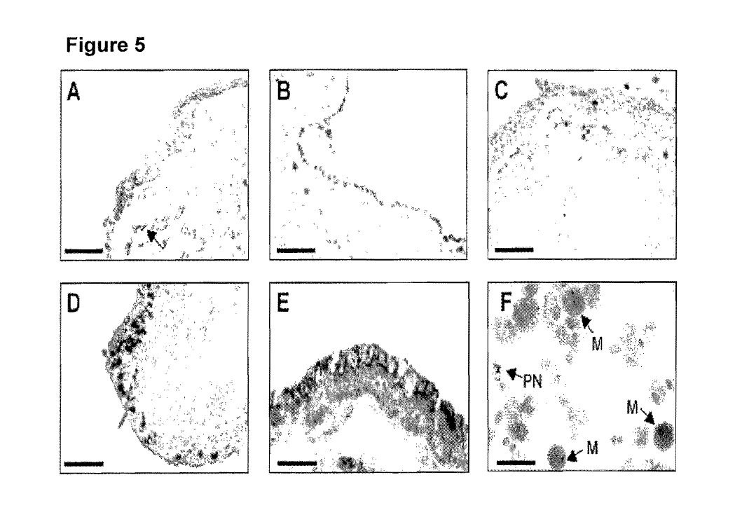

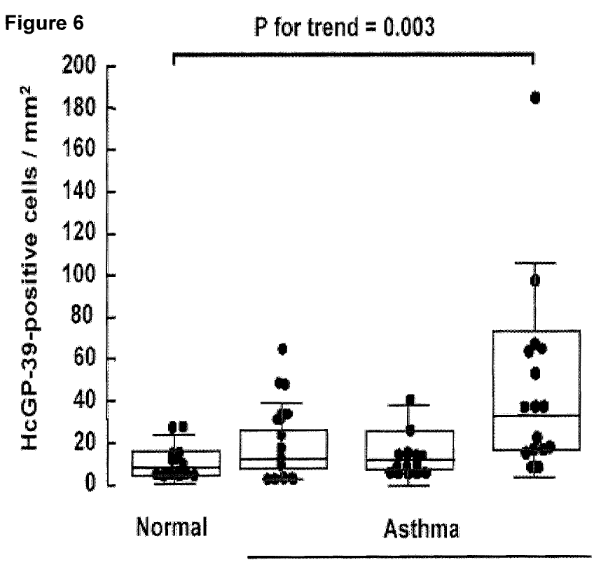

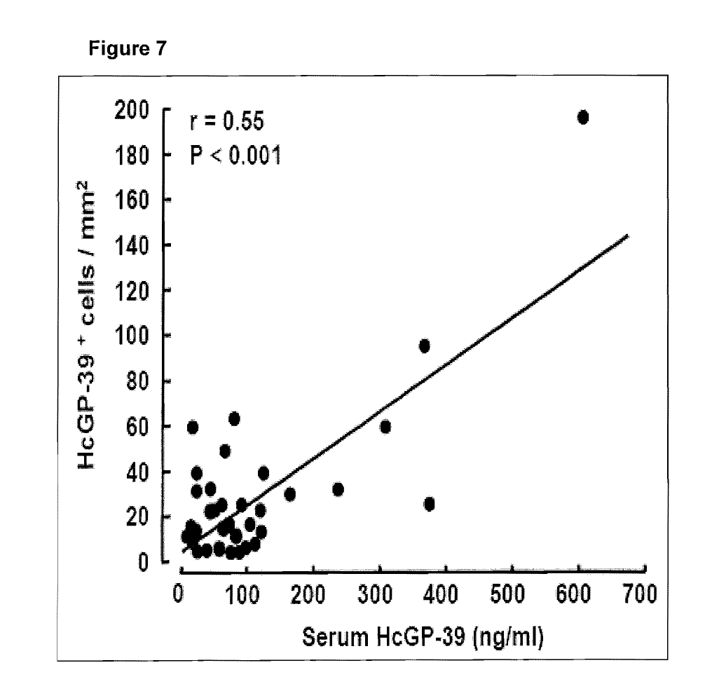

Based on the findings above, the expression of YKL-40 in bronchial biopsies from the Paris cohort was evaluated (FIG. 5). Bronchoscopy and bronchial biopsies were obtained from normals and asthmatics according to ATS guidelines. These studies included 12 normals, and 15, 10, and 15 patients with mild, moderate, and severe disease, respectively. These patients did not differ in terms of age or gender, but did differ in the levels of serum IgE, pulmonary function and doses of anti-asthma medications. IHC evaluations were undertaken with these biopsies using an affinity-purified monoclonal anti-YKL-40. These studies demonstrated that in control subjects there were rare YKL-40 expressing cells and in asthmatics the number was significantly increased [median (interquartile range), 3.1 positive cells mm² (2.1-7.4) versus 16.2 positive cells per mm² (9.1-30.2) in normals and asthmatics, respectively, p=0.005](data not shown). As shown in FIG. 5, YKL-40 staining was seen in subepithelial cells from the majority of asthmatics (FIG. 5B through FIG. 5E). In severe asthmatics the number of YKL-40 staining subepithelial cells was increased and staining of the bronchial epithelium was also evident (FIG. 5D and FIG. 5E). In BAL cytospin preparations from these asthmatics YKL-40 was found in the cytoplasm of macrophages and neutrophils (FIG. 5F). Importantly, in asthmatics, lung YKL-40 levels correlated with asthma severity and serum YKL-40 levels (r=0.548, p<0.001) (FIG. 6 and FIG. 7) No correlations were observed between the number of YKL-40 positive cells in biopsies and inflammatory cell (macrophages, eosinophils, lymphocytes, or neutrophils) numbers in BAL. Serum YKL-40 levels also correlated inversely with FEV₁ in all three cohorts (Yale, r=−0.22, P=0.01; Wisconsin, r=−0.33, P=0.009 and Paris, r=−0.21, P=0.005, data not shown). Thus, these studies demonstrate that the levels of circulating YKL-40 correlate with asthma severity, SBM thickness and pulmonary function in these patient cohorts

The relationship between asthma severity, SBM thickness and the levels of YKL-40 was also examined and showed that the SBM was thicker in mild and moderate asthmatics compared to controls (median [interquartile range], 9.4 μm [7.0-10.9], 9.2 μm [8.8-10.80] and 4.7 μm [3.9-4.9], P<0.001, P<0.001 respectively) and was thickest in the severe asthmatics ([12.4 μm [11.5-13.4], P<0.001, P<0.001, P=0.003 compared to normals, mild and moderate asthmatics respectively, data not shown).

Importantly, there was a significant correlation between SBM thickness and the serum YKL-40 levels in this population (r=0.51, P=0.003).

To further understand the patients with high levels of circulating YKL-40, a post-hoc analysis correlating serum YKL-40 levels and asthma characteristics in the Yale cohort demonstrated that YKL-40 levels correlated positively with the number of corticosteroid tapers in the last year, the dose of oral corticosteroids and the frequency of rescue inhaler use, and negatively with the percent predicted FEV₁. YKL-40 was not associated with history of atopy or IgE level. Multivariable analysis of the data was undertaken on the Yale cohort to determine if the correlation between YKL-40 and asthma severity persisted after adjustments for confounders that varied significantly among the asthma severity groups and affected YKL-40 levels including age, race, gender, history of atopy, BMI, and levels of serum IgE. In accord with the initial observations, this analysis demonstrated that asthma severity was associated with YKL-40 levels (adjusted P for trend=0.02) after adjustment for these factors.

### Example 2

### CHILL YKL-40, and Asthma Phenotpes in the Hutterites

### A. Subject and Patient Cohorts

### (1) The Hutterites

To minimize the confounding effects of genetic and environmental heterogeneity, genetic studies of disclosed herein focused on common diseases in the Hutterites (Ober et al., 2001, Am. J. Hum. Genet. 69:1068-1079; Ober et al., 2000, Am. J. Hum. Genet. 67:1154-1162]. The 753 Hutterites that were studied live on communal farms in South Dakota and are related to each other through multiple lines of descent in a 3028-person, 13-generation pedigree with 62 founders (Abney et al., 2001, Am. J. Hum. Genet. 68:1302-1307; Pan et al., 2007, Genet. Epidemiol. 31:338-347). The small number of founding genomes reduces genetic heterogeneity, and the communal lifestyle of the Hutterites ensures that nongenetic factors are remarkably uniform among persons. Smoking is prohibited (and rare) in this community, minimizing exposure to firsthand or secondhand smoke.

Asthma was assessed in 652 Hutterites by obtaining a history of symptoms (cough, wheeze, shortness of breath), bronchial hyperresponsiveness to methacholine inhalation or airway reversibility, and a doctor's diagnosis, according to previously published protocols (Ober et al., 2000, Am. J. Hum. Genet. 67:1154-1162; Lester et al., 2001, J. Allergy Clin. Immunol. 108:357-362). A total of 76 (11.7%) met the criteria for asthma; 80 others (12.3%) had bronchial hyperresponsiveness only, and 423 (64.9%) did not have bronchial hyperresponsiveness and were not symptomatic (Ober et al., 2000, Am. J. Hum. Genet. 67:1154-1162).

Persons were considered to have atopy if they had a positive skin-prick test for at least 1 of 14 airborne allergens (Ober et al., 2000, Am. J. Hum. Genet. 67:1154-1162); 311 of 702 Hutterites (44.3%) had atopy.

YKL-40 levels were measured in frozen serum specimens from 632 Hutterites who were 6 years of age or older (Chupp et al., 2007, N. Engl. J. Med. 357:2016-2027). The clinical characteristics of these 632 Hutterites are shown in Table 2.

TABLE 2
Baseline characteristics of the Hutterites with measured YKL-40 levels.*

| | | | |
|---|---|---|---|
| | Males | Females | Total |
| Characteristic | (N = 280) | (N = 352) | (N = 632) |

Age (year)

| | | | |
|---|---|---|---|
| Mean | 32.7 | 33.8 | 33.3 |
| Range | 6-92 | 6-88 | 6-92 |
| YLK-40 (ng/ml) | 96.7 ± 4.7 | 88.6 ± 3.5 | 92.2 ± 2.9 |
| Asthma no./no. | 36/251 (14.3) | 27/295 (9.2) | 63/546 (11.5) |
| tested; (%) | | | |
| Brochial | 63/251 (25.1) | 58/295 (19.7) | 121/546 (22.2) |
| hyperrespon. | | | |
| no./no. tested; | | | |
| (%) | | | |
| Atopy no./no. | 124/263 (47.1) | 116/320 (36.3) | 240/583 (41.2) |
| tested; (%) | | | |
| Serum IgE (IU) | 151.2 ± 21.7 | 49.8 ± 5.7 | 94.9 ± 10.3 |
| FEV₁ | 100.2 ± 1.0 | 101.2 ± 0.8 | 100.8 ± 0.6 |
| (% predicted value) | | | |
| FVC | 105.5 ± 0.9 | 106.5 ± 0.8 | 106.0 ± 0.6 |
| (% predicted value) | | | |
| FEV₁:FVC | 79.6 ± 0.5 | 82.2 ± 0.5 | 81.0 ± 0.4 |
| (% predicted value) | | | |
| FEF₂₅₋₇₅ | 3.7 ± 0.1 | 3.1 ± 0.1 | 3.3 ± 0.1 |
| (% predicted value) | | | |

*Plus-minus values are means ± SE.
Data for serum IgE were available for 610 Hutterites (271 males and 339 females).
Data for forced expiratory volume in 1 second (FEV₁), forced vital capacity (FVC), the FEV₁:FVC raio, and the forced expiratory flow between 25% and 75% of the FVC (FEF₂₅₋₇₅) were available for 599 Hutterites (272 males and 327 females).

For genetic studies, a natural-log transformation of the serum YKL-40 level was used to fulfill the distributional requirements of our methods, and we included age and sex as covariates. The heritability of YKL-40 levels was estimated with the use of variance-component methods (Abney et al., 2001, Am. J. Hum. Genet. 68:1302-1307; et al., 2007, Genet. Epidemiol. 31:338-347). As a test of association, the general two-allele model for quantitative measures was used (YKL-40 level, pulmonary-function measures, and total serum IgE level) (Abney et al., 2002, Am. J. Hum. Genet. 70:920-934).

Associations with binary phenotypes (asthma, bronchial hyperresponsiveness, and atopy) were assessed using the case-control quasi-likelihood test, which takes into account the relatedness between persons with the phenotypes and controls (Bourgain et al., 2003, Am. J. Hum. Genet. 73:612-626).

### (2) The Childhood Origins of Asthma Cohort

The Childhood Origins of Asthma (COAST) cohort consists of 206 children of European descent (56.8% of whom are boys) who participated in genetic studies in a birth-cohort study of the origins of asthma (Lemanske, 2002, Pediatr. Allergy Immunol. 13:Suppl 15:38-43), with asthma diagnosed at 6 years of age. Serum levels of YKL-40 were measured in 125 of these children at birth (in cord-blood specimens) and at 1 and 3 years of age and in 105 of these children at 5 years of age.

YKL-40 levels were measured in frozen serum specimens, according to the same protocols used for studies of the Hutterites (Chupp et al., 2007, N. Engl. J. Med. 357:2016-2027). At 6 years of age, the children in the COAST cohort received a diagnosis of asthma if they met at least one of the following criteria: doctor-diagnosed asthma, use of doctor-prescribed albuterol for episodes of coughing or wheezing more than once between 60 and 72 months of age, daily use of controller medication, implementation of a step-up plan as prescribed by a doctor (including the use of albuterol or the short-term use of inhaled corticosteroids during illness), or use of doctor-prescribed prednisone for the treatment of an asthma exacerbation.

The difference in YKL-40 levels between children with asthma and those without asthma were tested using a Wilcoxon rank-sum test. Associations between CHI3L1 SNPs and YKL-40 levels were examined with the use of log-transformed YKL-40 levels at birth and at 1, 3, and 5 years of age in a linear-regression model, with sex included as a covariate.

### (3) Asthma Case Patients and Controls

Two populations of European descent were used to replicate the associations with asthma. The Freiburg population consists of 344 children with asthma and 294 control children without asthma, recruited from clinics at the Children's University Hospital in Freiburg, Germany. Asthma was defined by the presence of self-reported symptoms (cough, wheeze, or shortness of breath), current use of asthma medications, a doctor's diagnosis, and bronchial hyperresponsiveness (i.e., a 15% decrease in the baseline value of the forced expiratory volume in 1 second [FEV₁] after either inhalation of ≦8 mg per deciliter of histamine or minutes of exercise). The controls did not have a history of asthma, recurrent wheezing, or atopy. A total of 64.7% of the case patients were male, with a mean age of 10.1 years (range, 6 to 16) at the time of evaluation; 59.4% of the controls were male, with a mean age of 7.9 years (range, 4 to 16) at the time of enrollment.

The Chicago population consisted of 99 case patients recruited through the adult and pediatric asthma clinics at the University of Chicago Medical Center and 197 controls recruited from the same medical center. Diagnosis of asthma in the case patients was based on fulfillment of all of the following criteria: age of 6 or more years, presence of at least two of three symptoms (cough, wheeze, and shortness of breath), a physician's diagnosis of asthma (with no conflicting pulmonary diagnosis), either bronchial hyperresponsiveness (defined as a ≧20% decrease in FEV₁ after inhalation of ≦25 mg of methacholine per milliliter) or an increase by 15% or more in FEV₁ after treatment with a short-acting bronchodilator or treatment with inhaled corticosteroids, and less than 3 pack-years of cigarette smoking (Lester et al., 2001, J. Allergy Clin. Immunol. 108:357-362). The controls were adults recruited from the University of Chicago Medical Center who did not have a history of asthma (either personally or among first-degree relatives). In all, 32.5% of the case patients were male, with a mean age of 24.4 years (range, 7 to 74) at the time of evaluation; 52.5% of the controls were male, with a mean age of 31.6 years (range, 18 to 69) at the time of evaluation.

Associations with asthma were tested with the use of Fisher's exact test for differences in the genotypes and allele frequencies between case patients and controls. The 95% confidence intervals were obtained from the hypergeometric distribution of the entries, conditional on fixed margins. The analysis for the two case-control populations combined was done using the Cochran-Mantel-Haenszel method.

### B. Genotyping and Quality Control Methods

### (1) Genotyping

The Hutterites in this study were genotyped with the Affymetrix GeneChip® 500 k Mapping Array, using both the early access and commercial Affymetrix GeneChip® 500 k Mapping Array at The University of Chicago. A set of 421,374 autosomal SNPs were present on both sets of chips. Another 1,423 nsSNPs were genotyped at the NHLBI Resequencing and Genotyping Service (Johns Hopkins University) using a custom 1,536 SNP oligo pool and BeadArray method, as previously described (Zhu et al., 2004, Science 304:1678-1682). In the combined set of SNPs, 131,049 were not further studied because either they were monomorphic (N=52,732) or had MAFs <5% (N=58,152) in the Hutterites. The remaining 310,490 SNPs were subjected to quality control checks. An additional 20,165 SNPs were excluded because either they had call rates <90% (N=3,614), they deviated from Hardy-Weinberg expectations at p<0.001 (correcting for the Hutterite inbreeding and population structure) (N=5,082), or because they generated >5 Mendelian errors (N=11,469), yielding a set of 290,325 markers with a median inter-maker spacing of 4.3 kb (range 17 bp-22.97 kb).

(2) Association testing in the Hutterites

The natural log of serum YKL-40 level was used for the heritability and association studies; age and sex were included as covariates in all analyses. The heritability of serum YKL-40 was estimated using a variance component maximum likelihood method. At each SNP, the general two-allele model test of association was used in the entire pedigree, keeping all inbreeding loops intact, as described. SNP-specific p-values were determined based on Gaussian theory; genome-wide p-values were determined by a Monte Carlo permutation-based test that preserves the covariance structure due to relatedness of individuals and assesses significance in the presence of multiple, dependent tests while guarding against deviations from normality in the data. We used 100 permutations to generate the empirical distribution of p-values and considered a p-value to be genome-wide significant if it was equal to or smaller than the 5% quantile of the permutation-based empirical distribution of the global minimum p value.

SNPs were then selected to tag all common haplotypes in CHI3L1 and within the 15 kb upstream of its transcriptional start site. Included in the analysis is the validated nonsynonymous SNP rs880633, the functional promoter SNP rs4950928 (Zhao et al., 2007, Am. J. Hum. Genet. 80:12-18), and a SNP (rs946263) previously shown to be associated with levels of expression of CHI3L1(Dixon et al., 2007, Nature Genet. 39:1202-1207). The tag SNPs rs4950928 (−131C→G), rs880633 (Arg145→Gly), rs10399805, rs1538372, and rs2275352 were genotyped using TaqMan Assay-on-Demand (ABI). An additional five SNPs in the 15-kb upstream region (including one tag SNP) were genotyped in specimens from the Hutterites, with the use of the Affymetrix GeneChip Mapping 500K Array; genotypes were determined by means of the BRLMM algorithm (Rabbee and Speed, 2006, Bioinformatics 22:7-12). Some redundant SNPs were included because they were present on the Affymetrix chip. The 10 SNPs were successfully genotyped in more than 95% of the persons studied, were in Hardy-Weinberg equilibrium (P>0.20), and in the Hutterites, had no mendelian errors. Allele frequencies and Hardy-Weinberg calculations for the Hutterites were adjusted for relatedness (Bourgain et al., 2004 Genetics 168:2349-2361; McPeek et al., 2004, Biometrics 60:359-367).

### Results:

Serum YKL-40 levels increased significantly with increasing age in the Hutterites (Pearson's r=0.21, P<0.001) but did not differ between males and females (t=0.52, P=0.61). Mean YKL-40 levels were increased among Hutterites with asthma (102.7 nanograms (ng) per milliliter) or bronchial hyperresponsiveness (96.5 ng per milliliter), as compared with controls (87.2 ng per milliliter) (P=0.005 and P=0.002, respectively), but as in our previous study (Chupp et al., 2007, N. Engl. J. Med. 357:2016-2027) the levels did not differ between subjects with atopy (99.4 ng per milliliter) and those without atopy (85.1 ng per milliliter) (P=0.68). Among the Hutterites, serum YKL-40 levels were significantly inversely correlated with FEV₁ (P=0.02) but not with forced vital capacity (FVC) (P=0.16), the FEV₁:FVC ratio (P=0.98), or forced expiratory flow between 25% and 75% of the FVC (FEF₂₅₋₇₅) (P=0.41).

To assess the relative contribution of genes to the variance in YKL-40 levels among subjects, we first estimated the heritability of the YKL-40 level. The narrow heritability (h²) of this trait in the Hutterites (±SE) is 0.51±0.10 and the broad heritability (H²) is 1.0±0.16. The high estimate for broad heritability indicates that differences in serum YKL-40 levels among individual Hutterites are due nearly entirely to genetic differences between individual persons. The comparatively large broad heritability indicates the presence of autosomal loci with significant non-additive (e.g., dominant) effects on YKL-40 levels.

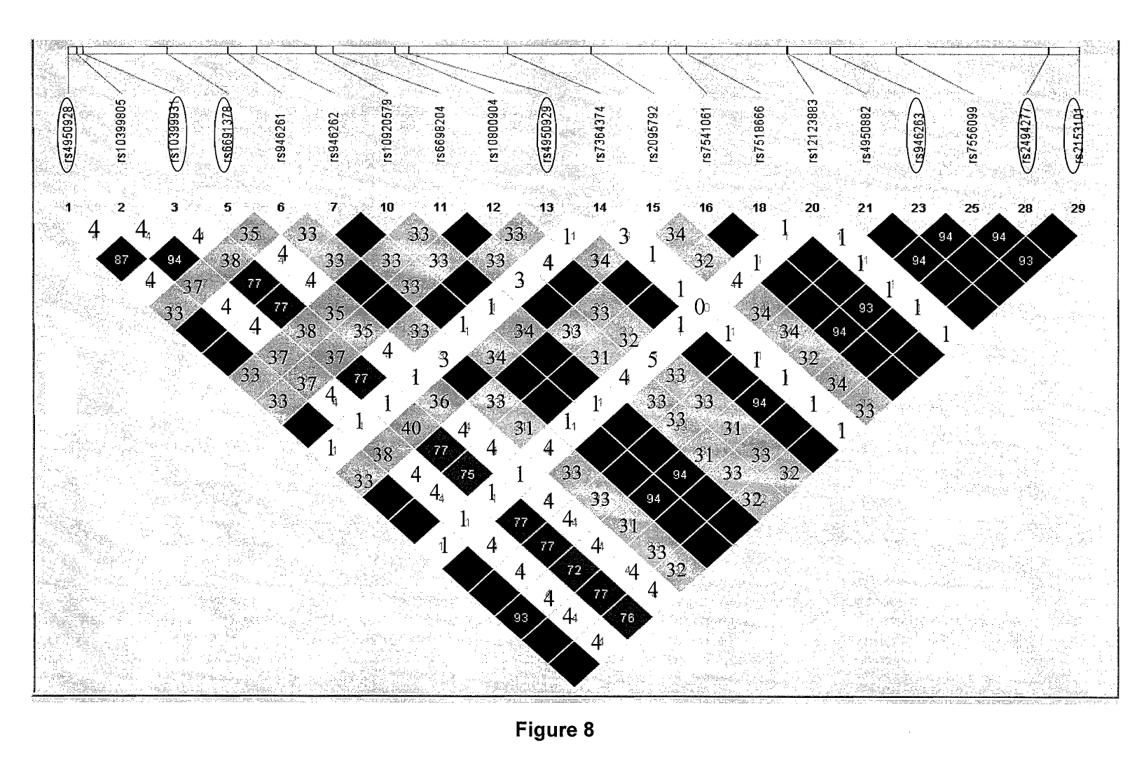

The most significant associations in the genome-wide association study were found between the YKL-40 level and SNPs upstream of the gene encoding YKL-40, CHI3L1 (FIG. 1C and FIG. 1D). The P values for all tested SNPs calculated with the use of the general two-allele model can be obtained from the National Institutes of Health Genotype and Phenotype database, dbGaP (wvvw.ncbi.nlm.nih.gov/entrez/query.fcgi?db=gap). To further evaluate the specific contribution of the CHI3L1 locus to the variance in YKL-40 levels, five additional SNPs were genotyped in the Hutterites; the location of these SNPs and the linkage-disequilibrium structure of the gene in this population are shown in FIG. 8. Three SNPs on the Affymetrix chip (rs4950929, rs946263, and rs2153101) are in perfect linkage disequilibrium with the functional promoter SNP −131C→G (rs4950928) (r²=1.0) (FIG. 8).

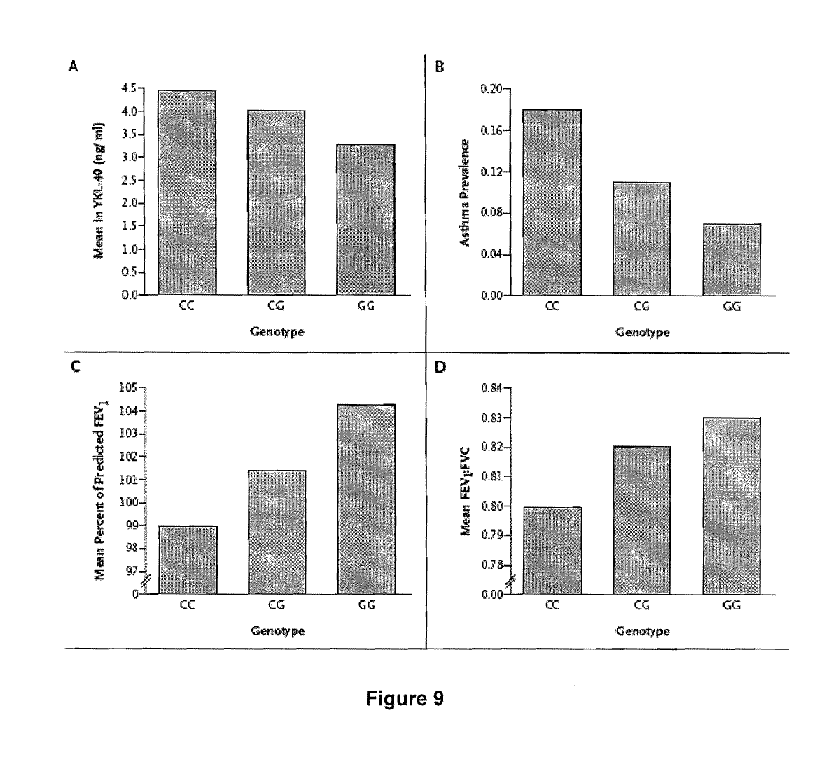

These four SNPs showed the strongest association with serum YKL-40 levels of all the SNPs tested in the Hutterites (P≦1.3×10⁻¹² for all four comparisons) (Table 3), and remained statistically significant after correction for the number of SNPs present on the Affymetrix chip. None of the 10 SNPs (Table 2) had significant sex-specific effects on serum YKL-40 levels. The nonsynonymous SNP in exon 5 (Arg145→Gly) was not significantly associated with YKL-40 levels (P=0.67) or any other phenotypic characteristic (Table 2). The major (most common) allele at each of the associated SNPs in CHI3L1 is the ancestral allele, according to the sequence of the orthologous gene in the chimpanzee. The −131C→G SNP rs4950928 accounts for 9.4% of the variance in YKL-40 levels in the Hutterites, with the minor G allele having an additive (negative) effect on YKL-40 levels (FIG. 9A).

TABLE 3A
Results of Association studies of single nucleotide polymorphisms
(SNPs) in the CHI3L1 gene and its upstream region on chromosome
1q32.1 among the Hutterites.*

| | Location relative to | Distance from | Minor |
|---|---|---|---|
| | Translational site | p terminus | allele |
| SNP | base pairs | | frequency |
| rs871799 | −14.120 | 201,436,494 | 0.18 |
| rs2153101 | −12723 | 201,435,097 | 0.21 |
| rs946263 | −9630 | 201,432,004 | 0.21 |
| rs4950929 | −4375 | 201,426,749 | 0.21 |
| rs6691378 | −1371 | 201,423,745 | 0.21 |
| rs10399805 (tag SNP) | −247 | 201,422,621 | 0.24 |
| rs4950928 (tag SNP) | −131 | 201,422,505 | 0.22 |
| rs1538372 (tag SNP) | +1220 (introns 2) | 201,421,155 | 0.38 |
| rs880633 (tag SNP)† | +2951 (exon 5) | 201,419,424 | 0.41 |
| rs2275352 (tag SNP) | +5573 (introns 7) | 201,4I6,802 | 0.24 |

*Associations were evaluated with use of the general two-allele model test for quantitative phenotypes (Abney et al., 2002, Am J Hum Genet 70: 920-934) and the case-control quasi-likelihood test (Bourgain et al., 2003, Am J Hum Genet 73: 612-626) for binary phenotypes. Allele frequencies are corrected for relatedness. Distances are based on build 126 of the National Center for Biotechnology Information's SNP database (dbSNP). FEF₂₅₋₇₅ denotes forced expiratory flow between 25% and 75% of the forced vital capacity (FVC), and the FEV₁, forced expiratory volume in 1 second.
†This SNP is the validated nonsynonomous SNP Arg145→Gly.

TABLE 3B
Results of Association studies of single nucleotide polymorphisms (SNPs) in the CHI3L1 gene and its
upstream region on chromosome 1q32.1 among the Hutterites.*
P values for Association

| | Serum | % | % | | | Total | | | |
|---|---|---|---|---|---|---|---|---|---|
| | YKL-40 | predicted | predicted | | | Serum | | Brochial | |
| SNP | level | FEV₁ | FVC | FEV₁:FVC | FEF₂₅₋₇₅ | IgE | Asthma | Hyperresp | Atopy |
| rs871799 | 0.03 | 0.29 | 0.23 | 0.99 | 0.83 | 0.68 | 0.86 | 0.68 | 0.93 |
| rs2153101 | 9.7 × 10⁻¹³ | 0.02 | 0.10 | 6.7 × 10⁻⁴ | 0.03 | 0.40 | 0.008 | 5.9 × 10⁻⁴ | 0.24 |
| rs946263 | 9.7 × 10⁻¹³ | 0.02 | 0.10 | 6.7 × 10⁻⁴ | 0.03 | 0.40 | 0.008 | 5.9 × 10⁻⁴ | 0.24 |
| rs4950929 | 1.3 × 10⁻¹² | 0.02 | 0.11 | 7.3 × 10⁻⁴ | 0.03 | 0.39 | 0.008 | 5.9 × 10⁻⁴ | 0.23 |
| rs6691378 | 3.8 × 10⁻⁵ | 0.03 | 0.02 | 0.59 | 0.59 | 0.68 | 0.82 | 0.46 | 0.54 |
| rs10399805 | 5.8 × 10⁻⁵ | 0.10 | 0.05 | 0.30 | 0.74 | 0.94 | 0.83 | 0.68 | 0.97 |
| (tag SNP) | | | | | | | | | |
| rs4950928 | 1.1 × 10⁻¹³ | 0.046 | 0.50 | 0.002 | 0.03 | 0.37 | 0.047 | 0.002 | 0.20 |
| (tag SNP) | | | | | | | | | |
| rs1538372 | 3.1 × 10⁻³ | 0.89 | 0.67 | 0.05 | 0.37 | 0.69 | 0.33 | 0.43 | 0.92 |
| (tag SNP) | | | | | | | | | |
| Rs880633 | 0.67 | 0.27 | 0.79 | 0.20 | 0.45 | 0.07 | 0.33 | 0.16 | 0.78 |
| (tag SNP)† | | | | | | | | | |
| Rs2275352 | 1.8 × 10⁻⁴ | 0.11 | 0.047 | 0.58 | 0.97 | 0.57 | 0.49 | 0.24 | 0.53 |
| (tag SNP) | | | | | | | | | |

*Associations were evaluated with use of the general two-allele model test for quantitative phenotypes (Abney et al., 2002, Am J Hum Genet 70: 920-934) and the case-control quasi-likelihood test (Bourgain et al., 2003, Am J Hum Genet 73: 612-626) for binary phenotypes. Allele frequencies are corrected for relatedness. Distances are based on build 126 of the National Center for Biotechnology Information's SNP database (dbSNP). FEF₂₅₋₇₅ denotes forced expiratory flow between 25% and 75% of the forced vital capacity (FVC), and the FEV₁, forced expiratory volume in 1 second.
†This SNP is the validated nonsynonomous SNP Arg145→Gly.

In the Hutterites, the frequency of the rs4950928 C allele was 0.84 in persons with asthma, 0.83 in persons with bronchial hyperresponsiveness, and 0.79 in controls; the allele was significantly associated with the asthma phenotype (P=0.047 by the case-control quasi-likelihood test) and the bronchial hyperresponsiveness phenotype (P=0.002 by the case-control quasi-likelihood test) (Table 3 and FIG. 9B). This SNP was not significantly associated with atopy (P=0.20 by the case-control quasi-likelihood test) or total serum IgE level (P=0.37 by the general two-allele model). The rs4950928 C allele was also a significant predictor of decreased FEV₁ (P=0.046 by the general two-allele model), decreased FEV₁:FVC (P=0.002 by the general two-allele model), and decreased FEF₂₅₋₇₅ (P=0.03 by the general two-allele model) in the Hutterites (Table 3 and FIGS. 9C and 9D).

### Example 3

### Replication Studies in the COAST Cohort

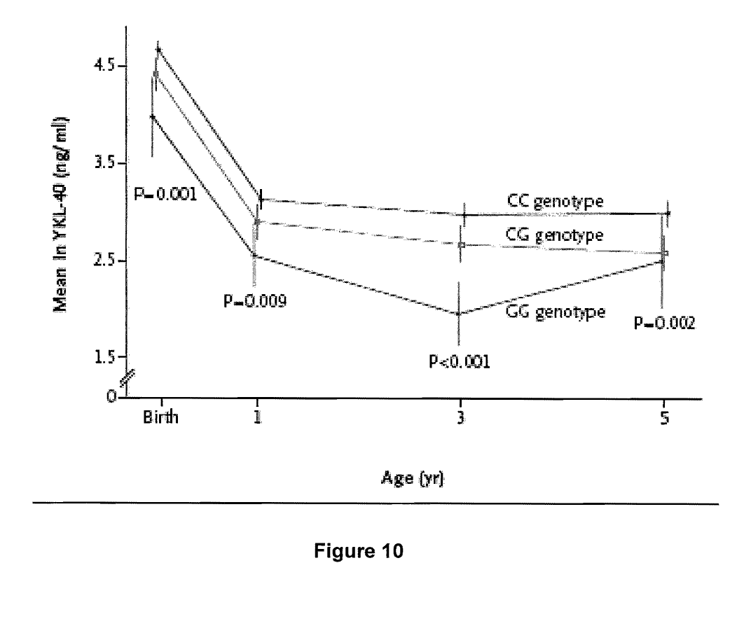

Serum YKL-40 levels were highest at birth and decreased through 3 years of age but were relatively stable between 3 and 5 years of age (FIG. 10, and Table 4). Serum YKL-40 levels at each age were not significant predictors of asthma diagnosis at 6 years of age, although the association at 3 years of age approached statistical significance (P=0.85 for the 121 subjects at birth, P=0.82 for the 121 subjects at 1 year of age, P=0.08 for the 121 subjects at 3 years of age, and P=0.29 for the 103 subjects at 5 years of age) (all P values by the Wilcoxon test).

TABLE 4
Mean in surem YLK-40 levels (ng/ml) in COAST children by age and CHI3L1-
131C→G genotype.

| | CC Genotype | | | CG Genotype | | | GG Genotype | | | |
|---|---|---|---|---|---|---|---|---|---|---|
| | N | Mean | SE | N | Mean | SE | N | Mean | SE | P-value |
| Cord blood | 82 | 4.66 | 0.048 | 39 | 4.41 | 0.082 | 4 | 3.98 | 0.207 | 0.0010 |
| Year 1 | 82 | 3.12 | 0.054 | 39 | 2.90 | 0.081 | 4 | 2.55 | 0.166 | 0.0089 |
| Year 3 | 82 | 2.97 | 0.063 | 39 | 2.67 | 0.092 | 4 | 1.95 | 0.169 | 0.00025 |
| Year 5 | 71 | 2.99 | 0.067 | 30 | 2.59 | 0.090 | 4 | 2.50 | 0.240 | 0.0016 |

N, sample size;
SE, standard error.

The −131C→G SNP (rs4950928) was also genotyped in the children in the COAST cohort. The −131C allele was associated with elevated YKL-40 levels at each age (FIG. 3), indicating that genotype-specific effects on circulating YKL-40 levels are present at birth and remain throughout the first 5 years of life. The changes among ages within genotype groupings were not significant. Among the 178 children whose asthma status was evaluated at 6 years of age, 52 (29.2%) received a diagnosis of asthma. The −131C→G genotype and allele frequencies did not differ significantly between children with asthma and those without asthma at 6 years of age. This result could be due to the different criteria (based on clinical criteria) used to diagnose asthma in the children in the COAST cohort, the influence of the CHI3L1 SNPs on YKL-40 levels before the onset of asthma-related sequelae, or the SNP having an independent effect on the risk of asthma later in life (i.e., after age 6).

### Example 4

### Replication Studies in the Case-Control Samples

In contrast to the COAST cohort, in the Freiburg population, the prevalence of the −131C allele was significantly greater in the case patients with asthma as compared with controls (frequency of the C allele, 0.81 among the case patients and 0.71 among the controls; P=1.6×10⁻⁴) (Table 3). In particular, the CC genotype was more common in patients with asthma (frequency, 0.66) than in controls (frequency, 0.51); both the CG and GG genotypes were more common among controls (CG frequency, 0.41 vs. 0.29 among the case patients; GG frequency, 0.08 vs. 0.05; P=5.6×10⁻⁴ by Fisher's exact test, assuming a dominant model). This pattern is similar to that found among the Hutterites. The odds ratio for the presence of one or two −131G alleles (CG or GG, vs. CC) was 0.54 (95% confidence interval [CI], 0.39 to 0.75), indicating that the minor −131G allele that is associated with reduced levels of circulating YKL-40 protein confers protection against asthma.

A similar pattern of association was present in the smaller Chicago population, in which the −131G allele was overrepresented in the controls as compared to the case patients (P=0.11 by the Fisher's exact test; P=0.03 by Fisher's exact test, assuming a dominant model; odds ratio for the G allele, 0.56; 95% CI, 0.32 to 0.95) (Table 3). The odds ratio for the G allele (CG or GG, vs. CC) in the two populations combined was 0.54 (95% CI, 0.41 to 0.71) (P=1.2×10⁻⁵ by the Cochran-Mantel-Haenszel method).

These data show that serum YKL-40 level is a highly heritable, quantitative trait in humans and confirms that YKL-40 level is a significant biomarker for asthma susceptibility and reduced lung function. Moreover, genetic variation in CHI3L1 influences serum YKL-40 levels and is associated with the risk of asthma, bronchial hyperresponsiveness, and reduced lung function. The SNP −131C→G SNP (rs4950928) seems likely to be the causal SNP; it is in the core promoter of CHI3L1, within a binding site for the MYC and MAX transcription factors. The minor allele (−131G on the forward strand) disrupts binding and was reported to be associated with reduced transcription in a luciferase reporter assay, lower messenger RNA levels in peripheral-blood cells, and reduced levels of circulating YKL-40 protein (Zhao et al., 2007, Am. J. Hum. Genet. 80:12-18). Furthermore, a SNP in strong linkage disequilibrium with −131C→G (rs946263) was found to influence CHI3L1 transcript levels in a genome-wide study of gene expression in cells from children with asthma (Dixon et al., 2007, Nature Genetics 39:1202-1207). The present data are consistent with these findings and indicate that the −131G allele is protective against asthma and decline in lung function, that this effect is independent of allergic (atopic) pathways, and that the effect of this SNP on circulating levels of YKL-40 is present at birth.

This and previous studies show an association between serum YKL-40 levels and a number of inflammatory conditions, (Johansen, 2006, Dan. Med. Bull. 53:172-209; Kruit et al., 2007, Respir. Med. 101:1563-1571) including asthma (Chupp et al., 2007, N. Engl. J. Med. 357:2016-2027), or between SNPs in CHI3L1 and serum YKL-40 levels (Kruit et al., 2007, Respir. Med. 101:1563-1571; Zhao et al., 2007, Am. J. Hum. Genet. 80:12-18) and gene expression (Zhao et al., 2007, Am. J. Hum. Genet. 80:12-18; Dixon et al., 2007, Nature Genetics 39:1202-1207), suggest that YKL-40 is an intermediate phenotype for asthma susceptibility. However, our results in the Hutterites and the COAST cohort do not allow us to reach this conclusion. For example, in the Hutterites, YKL-40 levels are not associated with the lung-function measures of FEV₁:FVC and FEF₂₅₋₇₅, yet the −131C→G SNP is a significant predictor of both (Table 2). In the COAST cohort, the −131C→G SNP is associated with YKL-40 levels at birth through 5 years of age but not with asthma at 6 years of age (FIG. 3). Thus, the possibility remains that variation in CHI3L1 exerts effects on the risk of asthma and on lung function that are independent of circulating levels of YKL-40.

In summary, an asthma susceptibility locus has been identified, CHI3L1, and showed that studying the genetics of quantitative traits (serum biomarkers) associated with asthma can identify asthma susceptibility loci. In the Hutterites, the CHI3L1 locus explains 9.4% of the variance in serum YKL-40 levels, suggesting that additional loci influence YKL-40 levels. Identifying the remaining loci that contribute to differences in serum YKL-40 levels and related proteins could identify additional genes with a significant effect on the risk of asthma and on lung function.

### Experiment 5

### Circulating Gene Expression Profiles, CHI3L1 Genotypes and Asthma Severity

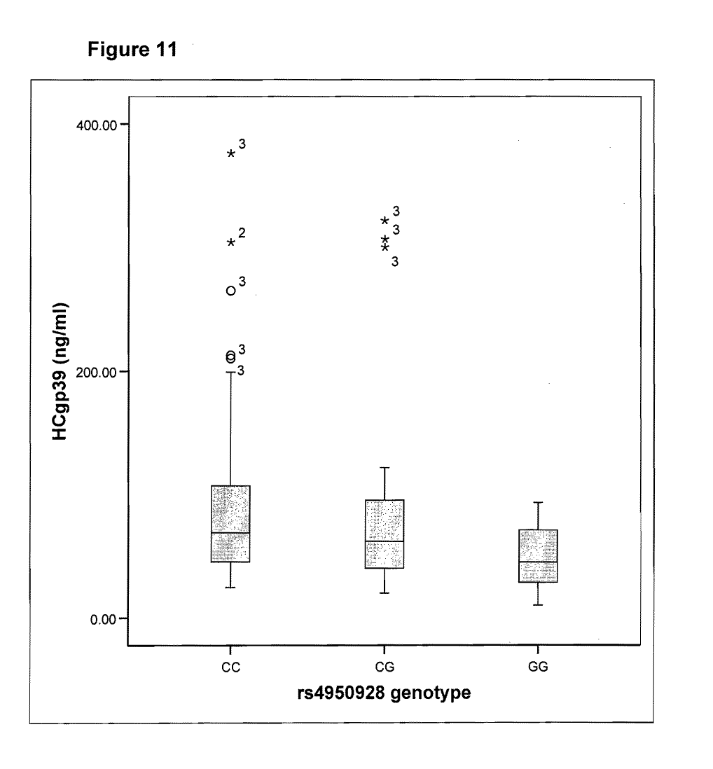

In an effort to understand the biologic differences that relate to CHI3L1/YKL-40 genotypes/phenotypes and asthma severity, the differences in these parameters and global gene expression in the YCAAD cohort, the NHLBI Severe Asthma Research Program (SARP) cohort, and the publicly accessible genome-wide association study of global gene expression dataset available online were examined. In the YCAAD population, the frequency of the rs4950928 G allele was 17% similar to the other populations. As expected YKL-40 levels correlated with the 131 CIG SNP (rs4950928) genotype (P for trend=0.036, FIG. 16). Importantly, nearly all of the patients with the highest levels of circulating YKL-40 have the CC genotype (points labeled with the number 3, FIG. 11). When the frequency of the CC genotype is examined as a function of severity it is clear that the frequency of the CC genotype is associated with greater asthma severity (mild 21%, moderate 33%, and severe 47%). This finding is consistent with the association of YKL-40 levels and asthma severity and suggest that gene expression will differ by rs4950928 genotype and this profile will correlate with asthma severity. Similar analysis of the SARP cohort shows other CHI3L1 polymorphisms correlate with asthma severity and lung function.

TABLE 5

| | CHI3L1 (209395_at) | | | |
|---|---|---|---|---|
| Official Symbol | rsID | Chr | LOD | p-value |
| RYR2 | rs4659902 | 1 | 3.312 | 9.40E−05 |
| LSAMP | rs3772958 | 3 | 3.308 | 9.50E−05 |
| RPS6KA2 | rs971152 | 6 | 2.499 | 0.00069 |
| | rs4709122 | 6 | 2.594 | 0.00055 |
| CPVL | rs10486610 | 76 | 2.9822.594 | 0.000210.00055 |
| | rs4709122 | | | |
| RSU1 | rs780637 | 10 | 2.476 | 0.00073 |
| GLT1D1CPVL | rs3843637 | 127 | 3.212.982 | 0.000120.00021 |
| | rs10486610 | | | |
| CLEC16ARSU1 | rs7185300 | 1610 | 2.3942.476 | 0.00090.00073 |
| | rs780637 | | | |
| GLT1D1 | rs3843637 | 12 | 3.21 | 0.00012 |
| CLEC16A | rs7185300 | 16 | 2.394 | 0.0009 |
| WWOX | rs16947192 | 16 | 2.423 | 0.00084 |

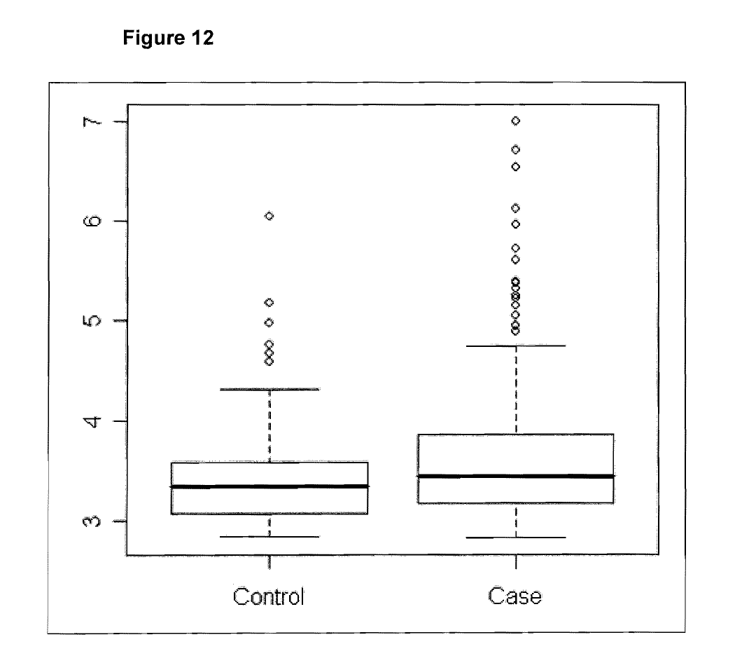

CHI3L1 gene expression was examined in the peripheral blood from a study for the expression QTL (eQTL) with 404 children with physician diagnosed asthma (mean age=9.62 yr for cases and 10.95 for controls) to evaluate the relationship between SNPs CHI3L1 circulating gene expression levels that is available on line. Genotyping was done with the Illumina Sentrix HumanHap 300 BeadChip, and the gene expression in lymphoblastoid cell line (EBVL) was measured with Affymetrix GeneChip Human Genome U133 Plus 2.0 Array. Most samples were collected from UK and Germany. The data were extracted from the database, mRNA by SNP Browser 1.0.1. As can be seen in FIG. 12, CHI3L1 mRNA expression is significantly in asthmatics (cases) than controls (P=0.04). In addition, when GWAS was done for associations with elevated levels of CHI3L1 gene expression there are many regions in the genome with high expression levels of CHI3L1 transcripts and the number of shared regions is much larger than by chance (with an expected overlapping region being 0.5) (Table 5).

### Experiment 6

### Predicting Patient Responsiveness to a Therapeutic Composition Administered for the Treatment of Asthma

Measuring YKL-40 levels in a subject diagnosed with or being treated for asthma is a useful tool in asthma management in terms of selecting patients that may respond to a particular therapeutic, as a biomarker of therapeutic response during treatment, or as a prognostic marker of future severity, risk of exacerbations, or decline in lung function.

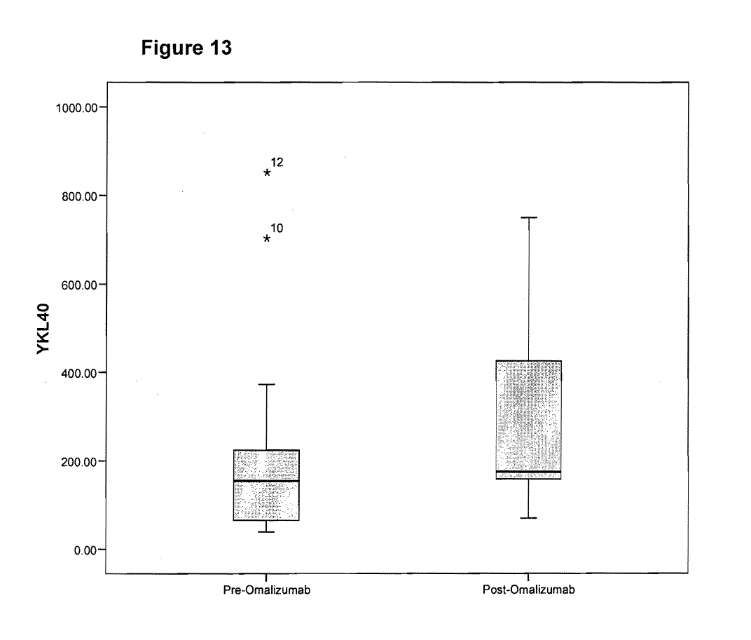

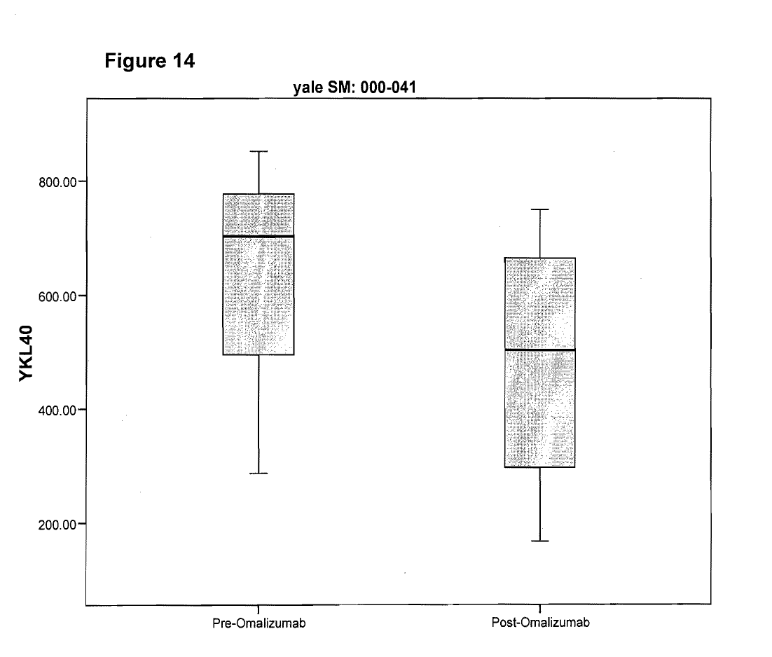

The most important therapy developed to treat severe asthma in the last 10 years is omalizumab, a humanized, monoclonal antibody directed against IgE. The relationship between YKL-40 levels and omalizumab therapy was examined in the asthma severity cohort followed in the Yale Center for Asthma and Airways Disease (YCAAD). These asthma patients from the New Haven area have consented to participate in the Mechanisms and Mediators of Asthma and COPD Longitudinal Study that has been ongoing in YCAAD for the last 8 years (Yale HIC#12268). From this population, 38 serum samples have been collected from 13 subjects that were treated with omalizumab. Most of these subjects are homozygous for the rs 4950928 CHI3L1 at risk genotype. Pre/post samples are available from only one patient. As can be seen in FIG. 13, the median YKL-40 level was 154 ng/ml in subjects prior to treatment with omalizumab. This is 2 fold higher compared to the levels we observed in the severe asthmatics (Chupp et al., NEJM) suggesting that YKL-40 levels are very high in patients that fail standard asthma therapies and are considered candidates for omalizumab. This suggests that Higher YKL-40 levels may be useful in identifying good omalizumab candidates. While patients on omalizumab treatment had slightly higher levels of YKL-40 (median 175 ng/ml), the one subject that had both pre and post omalizumab samples drawn had a 25% reduction in YKL-40 levels post Xolair treatment (FIG. 14). Clinically, this patient had a dramatic response to omalizumab therapy. Therefore, changing YKL-40 levels are a marker of omalizumab responsiveness. Finally, significant changes over time were observed in YKL-40 levels following initiation of omalizumab treatment suggesting that variations in the rate of change in YKL-40 level following initiation of omalizumab may prove useful as a biomarker as well.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying a human subject at-risk of developing a lung disorder, said method comprising obtaining a body sample from said subject; and, detecting at least one chromosomal variation in the CHI3L1 gene in said body sample, wherein if at least one chromosomal variation is detected in said gene, then said subject is at-risk of developing a lung disorder, wherein said lung disorder is selected from the group consisting of asthma, bronchial hyper-responsiveness, and reduced lung function.

2. The method of claim 1, wherein said body sample is selected from the group consisting of a tissue, a cell, and a bodily fluid.

3. The method of claim 1, wherein said detecting is performed using an assay selected from the group consisting of a PCR assay, a sequencing assay, an assay using a probe array, an assay using a gene chip, and an assay using a microarray.

4. The method claim 1, wherein said chromosomal variation is a −131 C→G in the promoter region of said CHI3L1 gene, defined by rs4950928 (SEQ ID NO:7).

5. A method of identifying a human subject at-risk of developing lung disorder, said method comprising: obtaining a body sample from said subject; detecting at least one disrupted transcript of the CHI3L1 gene in said body sample, wherein if at least one disrupted transcript is detected in said gene, then said subject is at-risk of developing said lung disorder, wherein said lung disorder is selected from the group consisting of asthma, bronchial hyperresponsiveness, and reduced lung function.

6. The method of claim 5, wherein said body sample is selected from the group consisting of a tissue, a cell, and a bodily fluid.

7. The method of claim 5, wherein said detecting is performed using an assay to assess the level of CHI3L1 mRNA, YKL-40 mRNA, or a combination thereof, in said body sample.

8. The method of claim 7, wherein said assay is selected from the group consisting of a Northern blot hybridization assay, an in situ hybridization assay, and a reverse transcriptase PCR assay.

9. The method of claim 5, wherein said detecting is performed using an assay to assess the level of CHI3L1 protein, YKL-40 protein, or a combination thereof, in said body sample.

10. The method of claim 9, where said assay is selected from the group consisting of a Western blot assay, a radioimmunoassay (RIA), an immunoassay, a chemiluminescent assay, and an enzyme-linked immunosorbent assay (ELISA).

11. A method of identifying a human subject afflicted with asthma likely to benefit from treatment with Omalizumab, said method comprising obtaining a body sample from said subject; and, detecting YKL-40 expression in said body sample, wherein if said YKL-40 expression in said sample is elevated relative to a control sample, then said subject is identified as likely to benefit from treatment with Omalizumab.

12. The method of claim 11, wherein said body sample is selected from the group consisting of a tissue, a cell, and a bodily fluid.

13. The method of claim 11, wherein said detecting is performed using an assay for YKL-40 mRNA.

14. The method of claim 13, wherein said assay is selected from the group consisting of a Northern blot hybridization assay, an in situ hybridization assay, and a reverse transcriptase PCR assay.

15. The method of claim 11, wherein said detecting is performed using an assay for YKL-40 protein.

16. The method of claim 15 where said assay is selected from the group consisting of a Western blot assay, a radioimmunoassay (RIA), an immunoassay, a chemiluminescent assay, and a enzyme-linked immunosorbent assay (ELISA).

17. A method of monitoring the efficacy of a therapeutic composition administered to a human subject for the treatment of asthma, said method comprising obtaining at least one body sample from said subject; and, detecting YKL-40 expression in said body sample, wherein if said YKL-40 expression in said sample remains elevated relative to a control sample after said composition is administered to said subject, then said composition is not efficacious for treating said subject.

18. The method of claim 17, wherein said body sample is selected from the group consisting of a tissue, a cell, and a bodily fluid.

19. The method of claim 17, wherein said detecting is performed using an assay for YKL-40 mRNA.

20. The method of claim 19, wherein said assay is selected from the group consisting of a Northern blot hybridization assay, an in situ hybridization assay, and a reverse transcriptase PCR assay.

21. The method of claim 17, wherein said detecting is performed in an assay for YKL-40 protein.

22. The method of claim 21, where said assay is selected from the group consisting of a Western blot assay, a radioimmunoassay (RIA), an immunoassay, a chemiluminescent assay, and an enzyme-linked immunosorbent assay (ELISA).

23. A method of identifying a human subject afflicted with a refractory lung disorder, said method comprising obtaining a body sample from said subject; and, detecting at least one chromosomal abnormality in the CHI3L1 gene in said body sample, wherein if at least one chromosomal abnormality is detected in said gene, then said subject is identified as having a refractory lung disorder, wherein said refractory lung disorder is selected from the group consisting of refractory asthma, refractory bronchial hyperresponsiveness, and refractory reduced lung function.

24. The method of claim 23, wherein said body sample is selected from the group consisting of a tissue, a cell, and a bodily fluid.

25. The method of claim 23, wherein said detecting is performed in an assay selected from the group consisting of a PCR assay, a sequencing assay, an assay using a probe array, an assay using a gene chip, and an assay using a microarray.

26. The method claim 23, wherein said chromosomal variation is a −131 C→G in the promoter region of said CHI3L1 gene, defined by rs4950928 (SEQ ID NO:7).

27. A method of identifying a human subject afflicted with a refractory lung disorder, said method comprising obtaining a body sample from said subject; and detecting at least one disrupted transcript of the CHI3L1 gene in said body sample, wherein if at least one disrupted transcript is detected in said gene, then said subject is identified as having a refractory lung disorder, wherein said refractory lung disorder is selected from the group consisting of refractory asthma, refractory bronchial hyperresponsiveness, and refractory reduced lung function.

28. The method of claim 27, wherein said body sample is selected from the group consisting of a tissue, a cell, and a bodily fluid.

29. The method of claim 27, wherein said detecting is performed in an assay for CHI3L1 mRNA, YKL-40 mRNA, or a combination thereof in said body sample.

30. The method of claim 29, wherein said assay is selected from the group consisting of a Northern blot hybridization assay, an in situ hybridization assay, and a reverse transcriptase PCR assay.

31. The method of claim 27, wherein said detecting is performed in an assay for CHI3L1 protein, YKL-40 protein or a combination thereof, in said body sample.

32. The method of claim 31, where said assay is selected from the group consisting of a Western blot assay, a radioimmunoassay (RIA), an immunoassay, a chemiluminescent assay, and an enzyme-linked immunosorbent assay (ELISA).
